(19) United States
(12) Patent Application Publication
(10) Pub. No.: US 2008/0045508 A1
(43) Pub. Date: Feb. 21, 2008

(54) SUBSTITUTED AMINOMETHYL BENZAMIDE COMPOUNDS

(76) Inventors: Brett Allison, Del Mar, CA (US); Nicholas I. Carruthers, Poway, CA (US); Michael P. Curtis, Brighton, MI (US); John M. Keith, San Diego, CA (US); Michael A. Letavic, San Diego, CA (US); Emily M. Stocking, Encinitas, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/766,153

(22) Filed: Jun. 21, 2007

Related U.S. Application Data
(60) Provisional application No. 60/806,167, filed on Jun. 29, 2006.

(51) Int. Cl. A61K 31/551; A61P 25/00; A61P 25/06; A61P 25/16; A61P 25/18; A61P 25/24; A61P 25/28; A61P 27/00; A61P 3/04; A61P 31/00; C07D 243/08; C07D 401/12

(52) U.S. Cl. 514/218; 540/575

(57) ABSTRACT

Certain substituted aminomethyl benzamide compounds are histamine H₃ receptor and/or serotonin transporter modulators useful in the treatment of histamine H₃ receptor- and/or serotonin-mediated diseases.

### CROSS-REFERENCE TO RELATED APPLICATIONS

This application claims the benefit of U.S. Provisional Application 60/806,167, filed Jun. 29, 2006, which is hereby incorporated by reference in its entirety.

### FIELD OF THE INVENTION

The present invention relates to certain aminomethyl benzamide compounds, pharmaceutical compositions containing them, and methods of using them for the treatment of disease states, disorders, and conditions mediated by the histamine H₃ receptor and/or the serotonin transporter.

### BACKGROUND OF THE INVENTION

The histamine H₃ receptor is primarily expressed in the mammalian central nervous system (CNS), with some minimal expression in peripheral tissues such as vascular smooth muscle. Several indications for histamine H₃ antagonists and inverse agonists have been proposed based on animal pharmacology and other experiments with known histamine H₃ antagonists (e.g. thioperamide). (See: “The Histamine H₃ Receptor-A Target for New Drugs”, Leurs, R. and Timmerman, H., (Eds.), Elsevier, 1998; Morisset, S. et al., Nature 2000, 408, 860-864.) These include conditions such as cognitive disorders, sleep disorders, psychiatric disorders, and other disorders.

Compounds that possess histamine H₃ receptor activity and serotonin transporter (SERT) activity may be useful in the treatment of SERT-mediated disorders such as substance abuse disorders and sexual dysfunction (including premature ejaculation), and particularly beneficial in the treatment of depression. Activation of the H₃ receptor on neurons by histamine or an agonist decreases the release of several neurotransmitters including noradrenaline and serotonin, key neurotransmitters involved in depression (Hill, S. J. et al. Pharmacol. Rev. 1997, 49(3), 253-278). Although H₃ receptor antagonists alone may not be capable of increasing serotonin levels in vivo to those required for antidepressant effects, concomitant blockade of the SERT will simultaneously decrease the neuronal reuptake of these neurotransmitter molecules, leading to enhanced concentrations of serotonin in the synaptic cleft and an enhanced therapeutic effect and a potentially reduced side effect profile as compared to a compound with SERT activity alone.

Histamine H₃ antagonists have been shown to have pharmacological activity relevant to several key symptoms of depression, including sleep disorders (e.g. sleep disturbances, fatigue, and lethargy) and cognitive difficulties (e.g. memory and concentration impairment), as described above. Therefore, a combined H₃/SERT modulating compound would provide symptomatic relief for the sleep disorders, fatigue, and cognitive problems during the first weeks of treatment, before the mood-elevating effect of the SERT modulation is noticed.

Compounds that have H₃ receptor activity and SERT activity have been disclosed in U.S. Pat. Publ. US 2006/0194837 A1 (published Aug. 31, 2006; based on U.S. patent application Ser. No. 11/300,880), U.S. Pat. Publ. US 2006/0293316 A1 (published Dec. 28, 2006; based on U.S. patent application Ser. No. 11/424,734), and U.S. Pat. Publ. US 2006/0287292 A1 (published Dec. 21, 2006; based on U.S. patent application Ser. No. 11/424,751), each of which is hereby incorporated by reference.

Aminomethyl benzamide ethers have been described by Pfizer, in Intl. Patent Appl. Publ. No. WO 01/72687, WO 02/18333, and U.S. Patent Appl. Publ. No. US 2002/0143003. Heteratom-linked aryl benzamides have been described by Glaxo SmithKline, in Intl. Patent Appl. Publ. No. WO 05/040144.

However, there remains a need for potent histamine H₃ receptor and/or serotonin transporter modulators with desirable pharmaceutical properties.

### SUMMARY OF THE INVENTION

Certain aminomethyl benzamide derivatives have now been found to have histamine H₃ receptor and/or serotonin transporter modulating activity. Thus, the invention is directed to the general and preferred embodiments defined, respectively, by the independent and dependent claims appended hereto, which are incorporated by reference herein.

In one general aspect the invention relates to a compound of the following Formula (I):

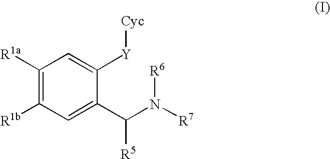

wherein
one of R^{1a} and R^{1b} is

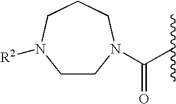

and the other is —H;
Y is —O—, —OCH₂—, —S—, —SO—, or —SO₂—;
R² is —H; a —C₁₋₆alkyl group unsubstituted or substituted with —OH, —OC₁₋₄alkyl, —NH₂, —NH(C₁₋₄alkyl), —N(C₁₋₄alkyl)₂, or —F; —CO₂C₁₋₄alkyl; or a monocyclic cycloalkyl group unsubstituted or substituted with —C₁₋₄alkyl, —OH, halo, or —CF₃;
R⁵ is —H or —C₁₋₆alkyl;
R⁶ is —H; or —C₁₋₆alkyl, —C₃₋₆alkenyl, —C₃₋₆alkynyl, monocyclic cycloalkyl, or —C₁₋₆alkyl-(monocyclic cycloalkyl), each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
R⁷ is —H; or —C₁₋₆alkyl, —C₃₋₆alkenyl, —C₃₋₆alkynyl, monocyclic cycloalkyl, —C₁₋₆alkyl-(monocyclic cycloalkyl), or —CO₂C₁₋₆alkyl, each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
or R⁶ and R⁷ taken together with their nitrogen of attachment form a saturated monocyclic heterocycloalkyl group unsubstituted or substituted with —C₁₋₄alkyl, —OH, —C₁₋₄alkyl-OH, —OC₁₋₄alkyl, or halo; and
Cyc is a phenyl or monocyclic carbon-linked heteroaryl group, unsubstituted or substituted with one, two, or three R^{k} moieties;
   where each R^{k} moiety is independently selected from the group consisting of: —C₁₋₆alkyl, —CHF₂, —CF₃, —C₂₋₆alkenyl, —C₂₋₆alkynyl, —OH, —OC₁₋₆alkyl, —OCHF₂, —OCF₃, —OC₃₋₆alkenyl, —OC₃₋₆alkynyl, —CN, —NO₂, —N(R^{l})R^{m}, —N(R^{l})C(O)R^{m}, —N(R^{l})SO₂C₁₋₆alkyl, —C(O)C₁₋₆alkyl, —S(O)₀₋₂—C₁₋₆alkyl, —C(O)N(R^{l})R^{m}, —SO₂N(R^{l})R^{m}, —SCF₃, halo, —CO₂H, and —CO₂C₁₋₆alkyl; or two R^{k} moieties on adjacent carbon atoms of attachment together are —OC₁₋₄alkyleneO— to form a cyclic ring which is unsubstituted or substituted with one or two fluoro substituents;
      where R^{l} and R^{m} are each independently —H or —C₁₋₆alkyl;
         or a pharmaceutically acceptable salt, pharmaceutically acceptable prodrug, or pharmaceutically active metabolite of such compound.

In a further general aspect, the invention relates to pharmaceutical compositions each comprising: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

In another general aspect, the invention is directed to a method of treating a subject suffering from or diagnosed with a disease, disorder, or medical condition mediated by histamine H₃ receptor and/or serotonin transporter activity, comprising administering to the subject in need of such treatment an effective amount of a compound of Formula (I), or a pharmaceutically acceptable salt, pharmaceutically acceptable prodrug, or pharmaceutically active metabolite thereof.

In certain preferred embodiments of the inventive method, the disease, disorder, or medical condition is selected from: cognitive disorders, sleep disorders, psychiatric disorders, and other disorders.

Additional embodiments, features, and advantages of the invention will be apparent from the following detailed description and through practice of the invention.

### DETAILED DESCRIPTION

The invention may be more fully appreciated by reference to the following description, including the following glossary of terms and the concluding examples. For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by /), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “alkylene” refers to a straight- or branched-chain alkyl group having from 1 to 12 carbon atoms in the chain, where two hydrogen atoms are removed to for a diradical. Examples of alkylene groups include methylene (—CH₂—), ethylene, n-propylene, isopropylene, butylene, and groups that in light of the ordinary skill in the art and the teachings provided herein would be considered equivalent to any one of the foregoing examples.

The term “alkenyl” refers to a straight- or branched-chain alkenyl group having from 2 to 12 carbon atoms in the chain. (The double bond of the alkenyl group is formed by two sp² hybridized carbon atoms.) Illustrative alkenyl groups include prop-2-phenyl, but-2-phenyl, but-3-phenyl, 2-methylprop-2-phenyl, hex-2-phenyl, and groups that in light of the ordinary skill in the art and the teachings provided herein would be considered equivalent to any one of the foregoing examples.

The term “alkynyl” refers to a straight- or branched-chain alkynyl group having from 2 to 12 carbon atoms in the chain. (The triple bond of the alkynyl group is formed by two sp hybridized carbon atoms.) Illustrative alkynyl groups include ethynyl, propynyl, butynyl, hexynyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

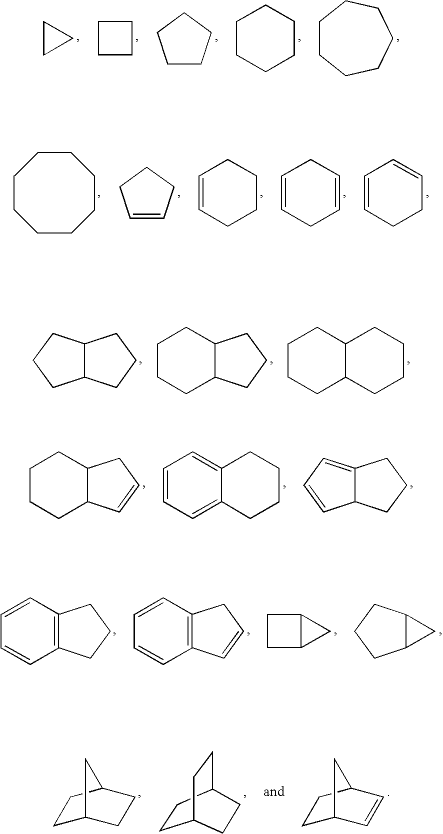

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative entities, in the form of properly bonded moieties, include:

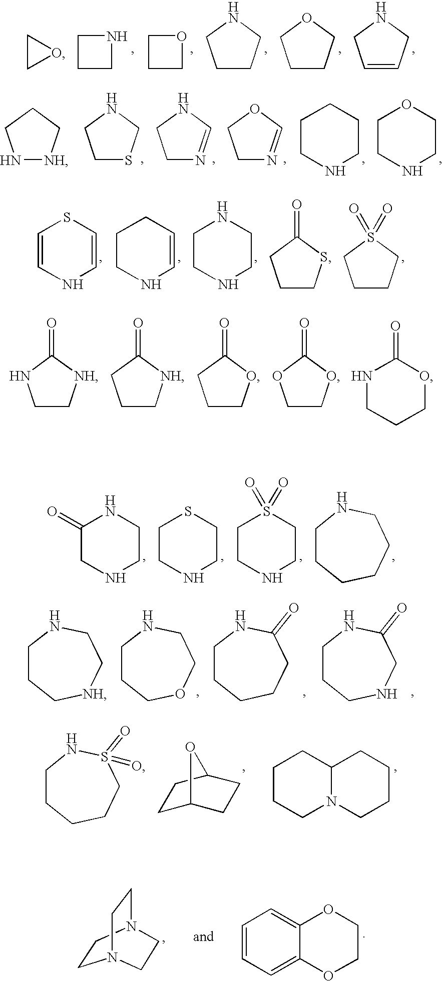

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

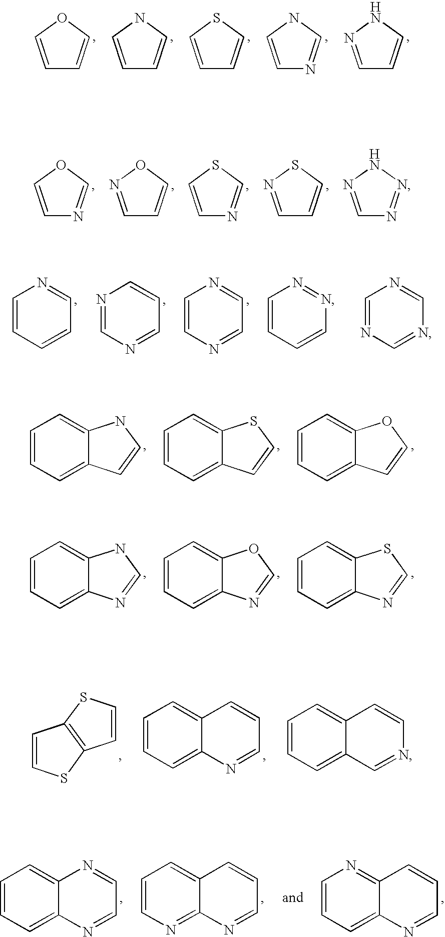

Those skilled in the art will recognize that the species of heteroaryl, cycloalkyl, and heterocycloalkyl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

In preferred embodiments of Formula (I), R^{1a} is

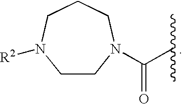

In preferred embodiments, Y is —O—. In other preferred embodiments, Y is —S—.

In preferred embodiments, R² is —H; or methyl, ethyl, propyl, isopropyl, sec-butyl, 2-methylpropyl, cyclopropyl, cyclobutyl, or cyclopentyl, each unsubstituted or substituted as previously described. In further preferred embodiments, R² is —H, methyl, ethyl, propyl, isopropyl, sec-butyl, 2-hydroxyethyl, 2-methoxyethyl, 2-dimethylaminoethyl, 2-hydroxy-2-methylpropyl, 3-dimethylaminopropyl, cyclopropyl, cyclobutyl, or cyclopentyl. In still further preferred embodiments, R² is —H, methyl, or cyclopropyl.

In preferred embodiments, R⁵ is —H or methyl. In further preferred embodiments, R⁵ is —H.

In preferred embodiments, R⁶ is —H, methyl, ethyl, isopropyl, sec-butyl, cyclopropyl, cyclobutyl, or cyclopentyl, each unsubstituted or substituted as previously described. In further preferred embodiments, R⁶ is —H, methyl, or methoxyethyl.

In preferred embodiments, R⁷ is —H, methyl, ethyl, propyl, isopropyl, sec-butyl, cyclopropyl, cyclobutyl, cyclopentyl, cyclohexyl, cyclopropylmethyl, cyclobutylmethyl, cyclopentylmethyl, or tert-butoxycarbonyl, each unsubstituted or substituted as previously described. In further preferred embodiments, R⁷ is methyl, ethyl, methoxyethyl, isopropyl, sec-butyl, cyclopropyl, cyclobutyl, or cyclopentyl. In still further preferred embodiments, R⁷ is methyl or cyclopropyl.

In alternative embodiments, R⁶ and R⁷ taken together with their nitrogen of attachment form azetidinyl, pyrrolidinyl, piperidinyl, piperazinyl, morpholinyl, thiomorpholinyl, 1,1-dioxo-1α⁶-thiomorpholin-4-yl, homopiperidinyl, diazepanyl, or homomorpholinyl, each unsubstituted or substituted as previously described. In further preferred embodiments, R⁶ and R⁷ taken together with their nitrogen of attachment form piperidinyl, pyrrolidinyl, morpholinyl, 2-hydroxymethyl-morpholin-4-yl, or homomorpholinyl.

In preferred embodiments, Cyc is a phenyl or pyridyl group unsubstituted or substituted with one, two, or three R^{k} moieties. In further preferred embodiments, Cyc is a thiophenyl, oxazolyl, thiazolyl, pyrazolyl, pyridinyl, or pyrazinyl group unsubstituted or substituted with one, two, or three R^{k} moieties.

In further preferred embodiments, Cyc is phenyl, 2-hydroxyphenyl, 3-hydroxyphenyl, 4-hydroxyphenyl, 4-hydroxy-2-methylphenyl, 4-hydroxy-3-fluorophenyl, 3,4-dihydroxyphenyl, 2-methoxyphenyl, 3-methoxyphenyl, 4-methoxyphenyl, 4-ethoxyphenyl, 2,4-dimethoxyphenyl, 2,5-dimethoxyphenyl, 3,4-dimethoxyphenyl, 3,5-dimethoxyphenyl, 3,4,5-trimethoxyphenyl, 2-methylphenyl, 3-methylphenyl, 4-methylphenyl, 4-ethylphenyl, 3-ethynylphenyl, 4-ethynylphenyl, 2-chlorophenyl, 3-chlorophenyl, 4-chlorophenyl, 2-fluorophenyl, 3-fluorophenyl, 4-fluorophenyl, 2-bromophenyl, 3-bromophenyl, 4-bromophenyl, 3-iodophenyl, 4-iodophenyl, 2,3-difluorophenyl, 2,4-difluorophenyl, 3,4-difluorophenyl, 2,3-dichlorophenyl, 2,4-dichlorophenyl, 2,5-dichlorophenyl, 3,4-dichlorophenyl, 3,5-dichlorophenyl, 2-fluoro-3-chlorophenyl, 2-fluoro-4-chlorophenyl, 2-chloro-4-fluorophenyl, 3-fluoro-4-chlorophenyl, 3-chloro-4-fluorophenyl, 4-fluoro-3-methylphenyl, 3-chloro-4-methoxyphenyl, 2-fluoro-4-methoxyphenyl, 3-fluoro-4-methoxyphenyl, 3-chloro-4-difluoromethoxyphenyl, 4-chloro-3-trifluoromethylphenyl, 2-trifluoromethylphenyl, 3-trifluoromethylphenyl, 4-trifluoromethylphenyl, 3-trifluoromethoxyphenyl, 4-trifluoromethoxyphenyl, 4-difluoromethoxyphenyl, 2-cyanophenyl, 3-cyanophenyl, 4-cyanophenyl, 3-acetylphenyl, 4-acetylphenyl, 3-nitrophenyl, 4-nitrophenyl, 4-aminophenyl, 4-dimethylaminophenyl, 4-carbamoylphenyl, 4-methanesulfanylphenyl, 4-methanesulfinylphenyl, 4-methanesulfonylphenyl, 4-trifluoromethanesulfanylphenyl, 3-methyl-4-methylsulfanylphenyl, benzo[1,3]dioxol-4-yl, benzo[1,3]dioxol-5-yl, thiophen-2-yl, thiophen-3-yl, oxazol-5-yl, thiazol-5-yl, thiazol-2-yl, 2H-pyrazol-3-yl, 2-pyridinyl, 3-pyridinyl, 4-pyridinyl, 4-trifluoromethyl-pyridin-2-yl, 2,6-dimethyl-pyridin-3-yl, 6-methyl-pyridin-3-yl, 2-chloro-5-pyridinyl, 2-dimethylamino-5-pyridinyl, 6-methoxy-pyridin-3-yl, 6-methylsulfanyl-pyridin-3-yl, 2-hydroxy-5-pyridinyl, 6-bromo-pyridin-3-yl, or pyrazin-2-yl.

In certain particular embodiments, Cyc is phenyl, 3-methoxyphenyl, 2-trifluoromethoxyphenyl, 2-fluorophenyl, 3-fluorophenyl, 4-fluorophenyl, 3-chlorophenyl, 4-chlorophenyl, 2,3-difluorophenyl, 2,3-dichlorophenyl, 3,4-dichlorophenyl, 2-chloro-4-fluorophenyl, 3-chloro-2-fluorophenyl, 4-chloro-2-fluorophenyl, 2-trifluoromethylphenyl, 4-trifluoromethylphenyl, 4-chloro-3-trifluoromethylphenyl, 4-methanesulfanylphenyl, 3-methyl-4-methanesulfanylphenyl, 4-trifluoromethanesulfanylphenyl, 4-trifluoromethyl-pyridin-2-yl, 2,6-dimethyl-pyridin-3-yl, 2-cyanophenyl, 3-cyanophenyl, 4-cyanophenyl, 2-pyridinyl, 3-pyridinyl, or 6-methyl-3-pyridinyl.

In preferred embodiments, each R^{k} moiety is selected from the group consisting of: methyl, fluoro, chloro, trifluoromethyl, methanesulfanyl, trifluoromethanesulfanyl, cyano, methoxy, and trifluoromethoxy.

In preferred embodiments, R^{l} and R^{m} are each independently —H or methyl.

In certain preferred embodiments, the compound of Formula (I) is selected from the group consisting of:

| Ex. | Compound Name |
|---|---|
| 1 | (4-Cyclopropyl-[1,4]diazepan-1-yl)-[4-(3,4-dichloro-phenoxy)-3- |
| | methylaminomethyl-phenyl]-methanone; |
| 2 | (4-Cyclopropyl-[1,4]diazepan-1-yl)-[3-methylaminomethyl-4-(4- |
| | trifluoromethyl-phenoxy)-phenyl]-methanone; |
| 3 | (4-Isopropyl-[1,4]diazepan-1-yl)-[3-methylaminomethyl-4-(4- |
| | trifluoromethyl-phenoxy)-phenyl]-methanone; |
| 4 | (4-Cyclopropyl-[1,4]diazepan-1-yl)-[3-methylaminomethyl-4- |
| | (pyridin-3-yloxy)-phenyl]-methanone; |
| 5 | [4-(4-Chloro-phenoxy)-3-methylaminomethyl-phenyl]-(4- |
| | cyclopropyl-[1,4]diazepan-1-yl)-methanone; |
| 6 | (4-Cyclopropyl-[1,4]diazepan-1-yl)-[4-(3-fluoro-phenoxy)-3- |
| | methylaminomethyl-phenyl]-methanone; |
| 7 | [3-Cyclopropylaminomethyl-4-(pyridin-3-yloxy)-phenyl]-(4- |
| | cyclopropyl-[1,4]diazepan-1-yl)-methanone; |
| 8 | [4-(4-Chloro-phenoxy)-3-cyclopropylaminomethyl-phenyl]-(4- |
| | cyclopropyl-[1,4]diazepan-1-yl)-methanone; |
| 9 | (4-Cyclopropyl-[1,4]diazepan-1-yl)-(3-methylaminomethyl-4- |
| | phenoxy-phenyl)-methanone; |
| 10 | [4-(3-Chloro-phenoxy)-3-methylaminomethyl-phenyl]-(4- |
| | cyclopropyl-[1,4]diazepan-1-yl)-methanone; |
| 11 | [4-(3-Chloro-phenoxy)-3-methylaminomethyl-phenyl]-(4-isopropyl- |
| | [1,4]diazepan-1-yl)-methanone; |
| 12 | (4-Cyclopropyl-[1,4]diazepan-1-yl)-[3-methylaminomethyl-4-(3- |
| | methyl-4-methylsulfanyl-phenoxy)-phenyl]-methanone; |
| 13 | (4-Cyclopropyl-[1,4]diazepan-1-yl)-[3-methylaminomethyl-4-(4- |
| | methylsulfanyl-phenoxy)-phenyl]-methanone; |
| 14 | [3-Cyclopropylaminomethyl-4-(3,4-dichloro-phenoxy)-phenyl]-(4- |
| | cyclopropyl-[1,4]diazepan-1-yl)-methanone; |
| 15 | [4-(3,4-Dichloro-phenoxy)-3-methylaminomethyl-phenyl]-(4- |
| | isopropyl-[1,4]diazepan-1-yl)-methanone; |
| 16 | [4-(3-Chloro-2-fluoro-phenoxy)-3-methylaminomethyl-phenyl]-(4- |
| | cyclopropyl-[1,4]diazepan-1-yl)-methanone; |
| 17 | [4-(3-Chloro-4-fluoro-phenoxy)-3-methylaminomethyl-phenyl]-(4- |
| | cyclopropyl-[1,4]diazepan-1-yl)-methanone; |
| 18 | [4-(4-Chloro-2-fluoro-phenoxy)-3-methylaminomethyl-phenyl]-(4- |
| | cyclopropyl-[1,4]diazepan-1-yl)-methanone; |
| 19 | (4-Cyclopropyl-[1,4]diazepan-1-yl)-[3-methylaminomethyl-4-(6- |
| | methyl-pyridin-3-yloxy)-phenyl]-methanone; |
| 20 | 4-[4-(4-Cyclopropyl-[1,4]diazepane-1-carbonyl)-2- |
| | methylaminomethyl-phenoxy]-benzonitrile; |
| 21 | [4-(4-Chloro-phenoxy)-3-cyclopropylaminomethyl-phenyl]-(4- |
| | isopropyl-[1,4]diazepan-1-yl)-methanone; |
| 22 | 3-[4-(4-Cyclopropyl-[1,4]diazepane-1-carbonyl)-2- |
| | methylaminomethyl-phenoxy]-benzonitrile; |
| 23 | [3-Cyclopropylaminomethyl-4-(3,4-dichloro-phenoxy)-phenyl]-(4- |
| | isopropyl-[1,4]diazepan-1-yl)-methanone; |
| 24 | (4-Cyclopropyl-[1,4]diazepan-1-yl)-[4-(4-fluoro-phenoxy)-3- |
| | methylaminomethyl-phenyl]-methanone; |
| 25 | [4-(2-Chloro-4-fluoro-phenoxy)-3-methylaminomethyl-phenyl]-(4- |
| | isopropyl-[1,4]diazepan-1-yl)-methanone; |
| 26 | (4-Cyclopropyl-[1,4]diazepan-1-yl)-[4-(3-methoxy-phenoxy)-3- |
| | methylaminomethyl-phenyl]-methanone; |
| 27 | (4-Cyclopropyl-[1,4]diazepan-1-yl)-[3-methylaminomethyl-4-(4- |
| | trifluoromethylsulfanyl-phenoxy)-phenyl]-methanone; |
| 28 | (4-Cyclopropyl-[1,4]diazepan-1-yl)-[3-methylaminomethyl-4-(2- |
| | trifluoromethoxy-phenoxy)-phenyl]-methanone; |
| 29 | 2-[4-(4-Cyclopropyl-[1,4]diazepane-1-carbonyl)-2- |
| | methylaminomethyl-phenoxy]-benzonitrile; |
| 30 | (4-Cyclopropyl-[1,4]diazepan-1-yl)-[3-methylaminomethyl-4-(2- |
| | trifluoromethyl-phenoxy)-phenyl]-methanone; |
| 31 | [4-(4-Chloro-3-trifluoromethyl-phenoxy)-3-methylaminomethyl- |
| | phenyl]-(4-cyclopropyl-[1,4]diazepan-1-yl)-methanone; |
| 32 | (4-Cyclopropyl-[1,4]diazepan-1-yl)-[4-(2,3-difluoro-phenoxy)-3- |
| | methylaminomethyl-phenyl]-methanone; |
| 33 | [4-(2-Chloro-4-fluoro-phenoxy)-3-methylaminomethyl-phenyl]-(4- |
| | cyclopropyl-[1,4]diazepan-1-yl)-methanone; |
| 34 | {4-(4-Chloro-phenoxy)-3-[(cyclopropyl-methyl-amino)-methyl]- |
| | phenyl}-(4-cyclopropyl-[1,4]diazepan-1-yl)-methanone; |
| 35 | (4-Cyclopropyl-[1,4]diazepan-1-yl)-[4-(2,3-dichloro-phenoxy)-3- |
| | methylaminomethyl-phenyl]-methanone; |
| 36 | (4-Cyclopropyl-[1,4]diazepan-1-yl)-[3-dimethylaminomethyl-4-(6- |
| | methyl-pyridin-3-yloxy)-phenyl]-methanone; |
| 37 | [4-(4-Chloro-phenylsulfanyl)-3-methylaminomethyl-phenyl]-(4- |
| | cyclopropyl-[1,4]diazepan-1-yl)-methanone; |
| 38 | (4-Cyclopropyl-[1,4]diazepan-1-yl)-[3-methylaminomethyl-4- |
| | (pyridin-2-ylsulfanyl)-phenyl]-methanone; |
| 39 | (4-Cyclopropyl-[1,4]diazepan-1-yl)-[3-methylaminomethyl-4- |
| | (pyridin-2-yloxy)-phenyl]-methanone; |
| 40 | [4-Cyclopropylaminomethyl-3-(pyridin-3-yloxy)-phenyl]-(4- |
| | cyclopropyl-[1,4]diazepan-1-yl)-methanone; |
| 41 | (4-Cyclopropyl-[1,4]diazepan-1-yl)-[4-piperidin-1-ylmethyl-3- |
| | (pyridin-3-yloxy)-phenyl]-methanone; |
| 42 | (4-Cyclopropyl-[1,4]diazepan-1-yl)-[4-(3,4-dichloro- |
| | benzyloxy)-3-methylaminomethyl-phenyl]-methanone; |
| 43 | (4-Isopropyl-[1,4]diazepan-1-yl)-[3-methylaminomethyl-4-(4- |
| | methylsulfanyl-phenoxy)-phenyl]-methanone; |
| 44 | (4-Cyclopropyl-[1,4]diazepan-1-yl)-[4-(2-fluoro-phenoxy)-3- |
| | methylaminomethyl-phenyl]-methanone; |
| 45 | [4-(4-Chloro-phenoxy)-3-methylaminomethyl-phenyl]-(4-isopropyl- |
| | [1,4]diazepan-1-yl)-methanone; |
| 46 | [4-(4-Chloro-3-trifluoromethyl-phenoxy)-3-methylaminomethyl- |
| | phenyl]-(4-isopropyl-[1,4]diazepan-1-yl)-methanone; |
| 47 | (4-Cyclopropyl-[1,4]diazepan-1-yl)-[3-methylaminomethyl-4-(4- |
| | trifluoromethyl-pyridin-2-ylsulfanyl)-phenyl]-methanone; |
| 48 | (4-Cyclobutyl-[1,4]diazepan-1-yl)-[4-(3,4-dichloro-phenoxy)-3- |
| | methylaminomethyl-phenyl]-methanone; |
| 49 | [4-Dimethylaminomethyl-3-(2,6-dimethyl-pyridin-3-yloxy)-phenyl]- |
| | (4-isopropyl-[1,4]diazepan-1-yl)-methanone; |
| 50 | (3-Benzyloxy-4-piperidin-1-ylmethyl-phenyl)-(4-isopropyl- |
| | [1,4]diazepan-1-yl)-methanone; |
| 51 | (4-Cyclopropyl-[1,4]diazepan-1-yl)-[4-morpholin-4-ylmethyl-3- |
| | (pyridin-3-yloxy)-phenyl]-methanone; |
| 52 | (4-Cyclopropyl-[1,4]diazepan-1-yl)-[3-(3,4-dichloro-phenoxy)-4- |
| | methylaminomethyl-phenyl]-methanone; |
| 53 | [4-(2-Hydroxymethyl-morpholin-4-ylmethyl)-3-phenoxy-phenyl]-(4- |
| | isopropyl-[1,4]diazepan-1-yl)-methanone; and |
| 54 | (3-Benzyloxy-4-{[bis-(2-methoxy-ethyl)-amino]-methyl}-phenyl)- |
| | (4-cyclopropyl-[1,4]diazepan-1-yl)-methanone; |

and pharmaceutically acceptable salts thereof.

The present invention also relates to a compound of Formula (I) that is a compound of the following Formula (II):

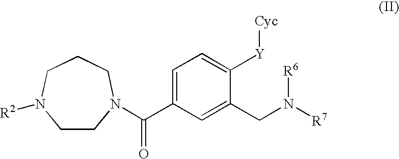

wherein

### Y is —O— or —S—;

and R², R⁶, R⁷, and Cyc are defined as for Formula (I);
or a pharmaceutically acceptable salt, pharmaceutically acceptable prodrug, or pharmaceutically active metabolite of such compound.

In preferred embodiments of Formula (II), Cyc is a phenyl or pyridyl group unsubstituted or substituted with one, two, or three R^{k} moieties.

The invention includes also pharmaceutically acceptable salts of the compounds represented by Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl)esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I), and uses of such metabolites in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al. J. Med. Chem. 1997, 40, 2011-2016; Shan, et al. J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites of the present invention are useful as modulators of the histamine H₃ receptor and/or the serotonin transporter in the methods of the invention. Accordingly, the invention relates to methods of using the compounds of the invention to treat subjects diagnosed with or suffering from a disease, disorder, or condition mediated by histamine H₃ receptor and/or serotonin transporter activity, such as those described herein.

The term “treat” or “treating” as used herein is intended to refer to administration of a compound or composition of the invention to a subject for the purpose of effecting a therapeutic or prophylacetic benefit through modulation of histamine H₃ receptor and/or serotonin transporter activity. Treating includes reversing, ameliorating, alleviating, inhibiting the progress of, lessening the severity of, or preventing a disease, disorder, or condition, or one or more symptoms of such disease, disorder or condition mediated through modulation of histamine H₃ receptor and/or serotonin transporter activity. The term “subject” refers to a mammalian patient in need of such treatment, such as a human. “Modulators” include both inhibitors and activators, where “inhibitors” refer to compounds that decrease, prevent, inactivate, desensitize or down-regulate histamine H₃ receptor and/or serotonin transporter expression or activity, and “activators” are compounds that increase, activate, facilitate, sensitize, or up-regulate histamine H₃ receptor and/or serotonin transporter expression or activity.

Accordingly, the invention relates to methods of using the compounds described herein to treat subjects diagnosed with or suffering from a disease, disorder, or condition mediated by histamine H₃ receptor and/or the serotonin transporter activity, such as: cognitive disorders, sleep disorders, psychiatric disorders, and other disorders. Symptoms or disease states are intended to be included within the scope of “medical conditions, disorders, or diseases.”

Cognitive disorders include, for example, dementia, Alzheimer's disease (Panula, P. et al., Soc. Neurosci. Abstr. 1995, 21, 1977), cognitive dysfunction, mild cognitive impairment (pre-dementia), attention deficit hyperactivity disorders (ADHD), attention-deficit disorders, and learning and memory disorders (Barnes, J. C. et al., Soc. Neurosci. Abstr. 1993, 19, 1813). Learning and memory disorders include, for example, learning impairment, memory impairment, age-related cognitive decline, and memory loss. H₃ antagonists have been shown to improve memory in a variety of memory tests, including the elevated plus maze in mice (Miyazaki, S. et al. Life Sci. 1995, 57(23), 2137-2144), a two-trial place recognition task (Orsetti, M. et al. Behav. Brain Res. 2001, 124(2), 235-242), the passive avoidance test in mice (Miyazaki, S. et al. Meth. Find. Exp. Clin. Pharmacol. 1995, 17(10), 653-658) and the radial maze in rats (Chen, Z. Acta Pharmacol. Sin. 2000, 21(10), 905-910). Also, in the spontaneously hypertensive rat, an animal model for the learning impairments in attention-deficit disorders, H₃ antagonists were shown to improve memory (Fox, G. B. et al. Behav. Brain Res. 2002, 131(1-2), 151-161).

Sleep disorders include, for example, insomnia, disturbed sleep, narcolepsy (with or without associated cataplexy), cataplexy, disorders of sleep/wake homeostasis, idiopathic somnolence, excessive daytime sleepiness (EDS), circadian rhythm disorders, fatigue, lethargy, jet lag, and REM-behavioral disorder. Fatigue and/or sleep impairment may be caused by or associated with various sources, such as, for example, sleep apnea, perimenopausal hormonal shifts, Parkinson's disease, multiple sclerosis (MS), depression, chemotherapy, or shift work schedules.

Psychiatric disorders include, for example, schizophrenia (Schlicker, E. and Marr, I., Naunyn-Schmiedeberg's Arch. Pharmacol. 1996, 353, 290-294), bipolar disorders, manic disorders, depression (Lamberti, C. et al. Br. J. Pharmacol. 1998, 123(7), 1331-1336; Perez-Garcia, C. et al. Psychopharmacology 1999, 142(2), 215-220) (Also see: Stark, H. et al., Drugs Future 1996, 21(5), 507-520; and Leurs, R. et al., Prog. Drug Res. 1995, 45, 107-165 and references cited therein.), obsessive-compulsive disorder, and post-traumatic stress disorder.

Other disorders include, for example, motion sickness, vertigo (e.g. vertigo or benign postural vertigo), epilepsy (Yokoyama, H. et al., Eur. J. Pharmacol. 1993, 234, 129-133), migraine, neurogenic inflammation, eating disorders (Machidori, H. et al., Brain Res. 1992, 590, 180-186), obesity, substance abuse disorders, tinitus, movement disorders (e.g. restless leg syndrome), eye-related disorders (e.g. macular degeneration and retinitis pigmentosis), and sexual dysfunction (including premature ejaculation).

Particularly, as modulators of the histamine H₃ receptor and/or the serotonin transporter, the compounds of the present invention are useful in the treatment or prevention of depression, disturbed sleep, narcolepsy, fatigue, lethargy, cognitive impairment, memory impairment, memory loss, learning impairment, attention-deficit disorders, and eating disorders.

In a treatment method according to the invention, an effective amount of a compound according to the invention is administered to a subject suffering from or diagnosed as having such a disease, disorder, or condition. An “effective amount” means an amount or dose sufficient to generally bring about the desired therapeutic or prophylacetic benefit in patients in need of such treatment.

Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of compound per kg of subject's body weight per day, preferably about 0.05 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylacetic effect is maintained. Of course, if symptoms have been alleviated to an appropriate level, treatment may cease. Patients may, however, require intermittent treatment on a long-term basis upon any recurrence of symptoms.

In addition, the compounds of the invention may be used in combination with additional active ingredients in the treatment of the above conditions. In an exemplary embodiment, additional active ingredients are those that are known or discovered to be effective in the treatment of conditions, disorders, or diseases mediated by histamine H₃ receptor and/or the serotonin transporter activity or that are active against another target associated with the particular condition, disorder, or disease, such as H₁ receptor antagonists, H₂ receptor antagonists, H₃ receptor antagonists, topiramate (Topamax™), and neurotransmitter modulators such as serotonin-norepinephrine reuptake inhibitors, selective serotonin reuptake inhibitors (SSRIs), noradrenergic reuptake inhibitors, non-selective serotonin re-uptake inhibitors (NSSRIs), acetylcholinesterase inhibitors (such as tetrahydroaminoacridine, Donepezil (Aricept™), Rivastigmine, or Galantamine (Reminyl™)), or modafinil. The combination may serve to increase efficacy (e.g., by including in the combination a compound potentiating the potency or effectiveness of a compound according to the invention), decrease one or more side effects, or decrease the required dose of the compound according to the invention.

More particularly, compounds of the invention in combination with modafinil are useful for the treatment of narcolepsy, excessive daytime sleepiness (EDS), Alzheimer's disease, depression, attention-deficit disorders, MS-related fatigue, post-anesthesia grogginess, cognitive impairment, schizophrenia, spasticity associated with cerebral palsy, age-related memory decline, idiopathic somnolence, or jet-lag. Preferably, the combination method employs doses of modafinil in the range of about 20 to 300 mg per dose.

The compounds of the invention are used, alone or in combination with one or more other active ingredients, to formulate pharmaceutical compositions of the invention. A pharmaceutical composition of the invention comprises: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a compound of the invention and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds of the invention may be prepared using suitable pharmaceutical excipients and compounding techniques now or later known or available to those skilled in the art. The compositions may be administered in the inventive methods by oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily.

Oral tablets may include a compound according to the invention mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil, sesame oil, or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the invention may utilize a patch formulation to affect transdermal delivery.

Compounds of the invention may alternatively be administered in methods of this invention by inhalation, via the nasal or oral routes, e.g., in a spray formulation also containing a suitable carrier.

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent.

Table of Acronyms and Abbreviations

| Term | Acronym or Abbreviation |
|---|---|
| tert-Butoxycarbonyl | Boc |
| 1,8-Diazabicyclo[5.4.0]undec-7-ene | DBU |
| 1,2-Dichloroethane | DCE |
| Dichloromethane | DCM |
| Diethyl azodicarboxylate | DEAD |
| Diisopropyl azodicarboxylate | DIAD |
| Ethylene glycol dimethyl ether | DME |
| N,N-Dimethylformamide | DMF |
| Diethyl ether | Et₂O |
| Ethyl acetate | EtOAc |
| Ethanol | EtOH |
| Methanol | MeOH |
| Trifluoroacetic acid | TFA |
| Tetrahydrofuran | THF |

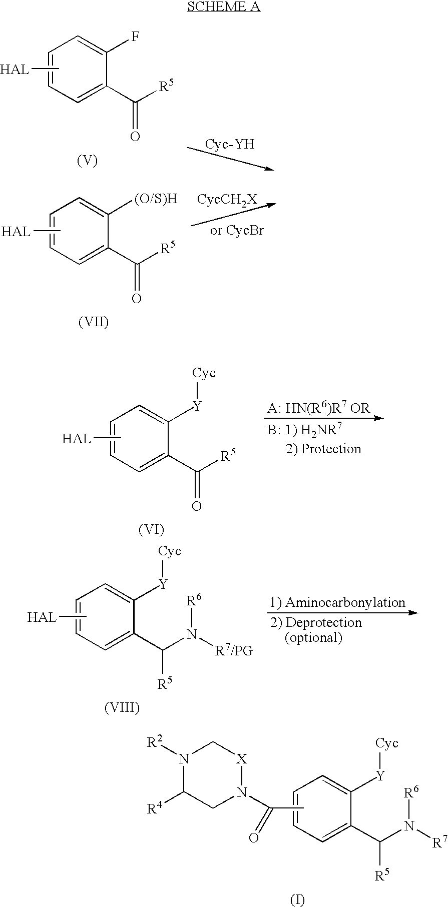

Referring to Scheme A, fluorobenzenes (V) and halobenzenes (VII), where HAL is Br or Cl, are commercially available or are prepared according to methods known to one skilled in the art. Fluorobenzenes (V) may be reacted with Cyc-YH (where Y is —O— or —S—) under aromatic substitution conditions to form ethers or thioethers (VI), where Y is —S— or —O—, in the presence of a suitable base such as K₂CO₃, Na₂CO₃, or Cs₂CO₃, in a solvent such as DMF, DME, or toluene, or a mixture thereof, at temperatures between room temperature and the reflux temperature of the solvent, to give ethers and thioethers (VI). Alkylation of phenols and thiols (VII) with CycCH₂X (where X is a suitable leaving group, such as Br, Cl, OTs, or the like), in the presence of a suitable base such as K₂CO₃, Na₂CO₃, NaH, or the like, in a solvent such as CH₃CN or THF, provides ethers or thioethers (VI). In another embodiment, phenols or thiols (VII) may be reacted under Mitsunobu conditions with CycCH₂X, in the presence of PPh₃ and DEAD or DIAD, in a solvent such as CH₃CN or THF, to form ethers or thioethers (VI), where Y is —OCH₂—. Aromatic substitution with activated CycBr reagents (where Cyc is a suitable heteroaryl group) may be accomplished in the presence of a suitable base such as K₂CO₃, Na₂CO₃, or Cs₂CO₃, in the presence of dehydryating agents such as molecular sieves or Ca₂O or a mixture thereof, and salicylaldoxime, in a solvent such as DMF, DME, or toluene, or a mixture thereof, at temperatures between room temperature and the reflux temperature of the solvent, to form ethers or thioethers (VI) where Y is —O— or —S—.

Ethers or thioethers (VI) may then be converted to benzyl amines (VIII) under reductive amination conditions known to one skilled in the art. Preferred conditions include a reducing agent such NaBH₄, NaCNBH₃, or NaBH(OAc)₃, in a solvent such as MeOH, EtOH, or DCE, and with optional additives such as acetic acid or a Lewis acid. Where a primary amine H₂NR⁷ is used for the reductive amination, the resulting benzyl amine may be protected in a subsequent step with a suitable nitrogen protecting group, such as a Boc or other suitable carbamoyl group, under conditions known to one skilled in the art.

Aminocarbonylation of benzyl amines (VIII) with suitable amines to give amides (I) may be performed in the presence of a suitable catalyst, such as Hermann's catalyst (trans-di-μ-acetatobis[2-(di-o-tolylphosphino)benzyl]-dipalladium), coupling aids such as tri-t-butylphosphonium tetrafluoroborate, a CO equivalent such as Mo(CO)₆, a suitable base such as DBU, in a solvent such as THF or toluene, at temperatures between room temperature and 150° C. in a microwave reactor. If a nitrogen protecting group is used, removal is then accomplished under conditions known in the art, such as acidic or hydrogenation conditions, following the coupling step.

Where the synthesis provides compounds where Y is —S—, oxidation to the corresponding sulfoxides and sulfones (Y is —SO— or —SO₂—) may be performed under conditions known in the art.

Those skilled in the art will recognize that several of the chemical transformations described above may be performed in a different order than that depicted in the above Schemes.

Additional applicable methodologies are described in U.S. Pat. Publ. US 2006/0194837 A1, U.S. Pat. Publ. US 2006/0293316 A1, and U.S. Pat. Publ. US 2006/0287292 A1.

Compounds of Formula (I) may be converted to their corresponding salts using methods known to those skilled in the art. For example, amines of Formula (I) may be treated with trifluoroacetic acid, HCl, or citric acid in a solvent such as Et₂O, CH₂Cl₂, THF, or MeOH to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following examples are provided to further illustrate the invention and various preferred embodiments.

### EXAMPLES

### Chemistry:

Where solutions or mixtures are “concentrated”, they are typically concentrated under reduced pressure using a rotary evaporator.

Normal phase flash column chromatography (FCC) was typically performed with RediSep® silica gel columns using 2 M N₃ in MeOH/DCM as eluent, unless otherwise indicated.

Preparative Reversed-Phase high performance liquid chromatography (HPLC) was typically performed using a Gilson® instrument with a YMC-Pack ODS-A, 5 μm, 75×30 mm column, a flow rate of 25 mL/min, detection at 220 and 254 nm, with a 15% to 99% acetonitrile/water/0.05% TFA gradient.

Analytical Reversed-Phase HPLC was typically performed using 1) a Hewlett Packard Series 1100 instrument with an Agilent ZORBAX® Bonus RP, 5 μm, 4.6×250 mm column, a flow rate of 1 mL/min, detection at 220 and 254 nm, with a 1% to 99% acetonitrile/water/0.05% TFA gradient; or 2) a Hewlett Packard HPLC instrument with an Agilent ZORBAX® Eclipse XDB-C8, 5 μm, 4.6×150 mm column, a flow rate of 1 mL/min, detection at 220 and 254 nm, with a 1% to 99% acetonitrile/water/0.05% TFA gradient.

Where trifluoroacetic acid salts were obtained, they were obtained from preparative reversed-phase HPLC or from deprotection of a Boc group with TFA in a final step. Where hydrochloride salts were obtained, they were obtained by treatment of a solution of the corresponding free base in DCM with an excess of 2.5 M HCl in MeOH, and concentration of the reaction solution.

In obtaining the characterization data described in the examples below, the following analytical protocols were followed as indicated.

Mass spectra were obtained on an Agilent series 1100 MSD using electrospray ionization (ESI) in either positive or negative modes as indicated. Calculated mass corresponds to the exact mass.

NMR spectra were obtained on either a Bruker model DPX400 (400 MHz), DPX500 (500 MHz), DRX600 (600 MHz) spectrometer. The format of the ¹H NMR data below is: chemical shift in ppm down field of the tetramethylsilane reference (multiplicity, coupling constant J in Hz, integration).

Chemical names were generated using ChemDraw Ultra 6.0.2 (CambridgeSoft Corp., Cambridge, Mass.).

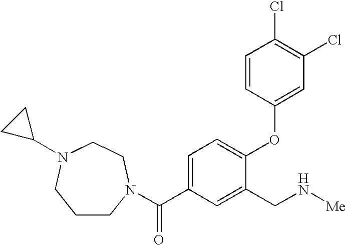

### Example 1

### (4-Cyclopropyl-[1,4]diazepan-1-yl)-[4-(3,4-dichloro-phenoxy)-3-methylaminomethyl-phenyl]-methanone

Step A: 5-Bromo-2-(3,4-dichloro-phenoxy)-benzaldehyde. To a solution of 5-bromo-2-fluoro-benzaldehyde (5.13 g, 25.4 mmol) in DMF (25 mL) were added K₂CO₃ (7.15 g, 51.8 mmol) and 3,4-dichloro-phenol (4.67 g, 28.8 mmol). The mixture was heated at 90° C. for 24 h and then was allowed to cool to room temperature (rt). Water was added and the mixture was extracted with Et₂O. The combined organic layers were dried (MgSO₄) and concentrated. The residue was diluted with DCM and hexanes and the resulting solids were collected by vacuum filtration to provide the desired product (4.74 g, 54%). ¹H NMR (CDCl₃): 10.36 (s, 1H), 8.06 (d, J=2.5, 1H), 7.67 (dd, J=8.8, 2.6, 1H), 7.46 (d, J=8.8, 1H), 7.17 (d, J=2.8, 1H), 6.92 (dd, J=8.8, 2.8, 1H), 6.84 (d, J=8.8, 1H).

Step B: [5-Bromo-2-(3,4-dichloro-phenoxy)-benzyl]-methyl-amine. To a mixture of 5-bromo-2-(3,4-dichloro-phenoxy)-benzaldehyde (4.74 g, 13.8 mmol) in MeOH (250 mL) was added MeNH₂ (40% aq.; 20 mL, 260 mmol), and the resulting mixture was stirred at rt until homogeneous. The mixture was cooled to 0° C. and treated with NaBH₄ (1.05 g, 27.8 mmol) portionwise. After 24 h, the mixture was concentrated and the residue was diluted with 1 N NaOH and extracted with DCM. The combined organic layers were dried (Na₂SO₄) and concentrated. The crude product was purified by FCC to provide the desired product (4.80 g, 97%). MS (ESI): mass calcd. for C₁₄H₁₂BrCl₂NO, 358.95; m/z found, 360.1 [M+H]⁺. ¹H NMR (CDCl₃): 7.61 (d, J=2.5, 1H), 7.40-7.37 (m, 2H), 7.03 (d, J=2.8, 1H), 6.82-6.79 (m, 2H), 3.72 (s, 2H), 2.44 (s, 3H), 1.30-1.21 (m, 1H).

Step C: [5-Bromo-2-(3,4-dichloro-phenoxy)-benzyl]-methyl-carbamic acid tert-butyl ester. To a solution of [5-bromo-2-(3,4-dichloro-phenoxy)-benzyl]-methyl-amine (4.61 g, 12.8 mmol) in DCM (250 mL) were added Et₃N (3.6 mL, 25.8 mmol) and di-tert-butyl dicarbonate (3.44 g, 15.8 mmol). After 1 h, the mixture was diluted with 1 N NaOH and extracted with DCM. The combined organic layers were dried (Na₂SO₄) and concentrated. The crude material was carried forward without purification (6.35 g, >100%). ¹H NMR (CDCl₃): 7.47-7.31 (m, 3H), 7.03 (d, J=2.8, 1H), 6.80-6.74 (m, 2H), 4.46-4.32 (m, 2H), 2.93-2.78 (m, 3H), 1.45 (br s, 9H).

Step D. [5-(4-Cyclopropyl-[1,4]diazepane-1-carbonyl)-2-(3,4-dichloro-phenoxy)-benzyl]-methyl-carbamic acid tert-butyl ester. To a solution of 5-bromo-2-(3,4-dichloro-phenoxy)-benzyl]-methyl-carbamic acid tert-butyl ester (304.0 mg, 0.62 mmol) in THF (2.5 mL) were added DBU (0.28 mL, 1.9 mmol), 1-cyclopropyl-[1,4]diazepane (262.5 mg, 1.9 mmol), Hermann's catalyst (23.4 mg, 0.025 mmol), tri-t-butylphosphium tetrafluoroborate (23.4 mg, 0.05 mmol), and Mo(CO)₆ (149 mg, 0.56 mmol). After 6 min in a microwave reactor at 125° C., the mixture was cooled to rt and concentrated. Purification by FCC gave the desired product, which was carried directly into the next step. MS (ESI): mass calcd. for C₂₈H₃₅Cl₂N₃O₄, 547.20; m/z found, 548.3 [M+H]⁺. ¹H NMR (CDCl₃): 7.42-7.28 (m, 3H), 7.06 (d, J=2.7, 1H), 6.93-6.89 (m, 1H), 6.82 (dd, J=8.8, 2.9, 1H), 4.52-4.42 (m, 2H), 3.80-3.72 (m, 2H), 3.52-3.42 (m, 2H), 2.96 (t, J=4.7, 1H), 2.92-2.75 (m, 6H), 2.00-1.74 (m, 3H), 1.50-1.38 (m, 9H), 0.54-0.32 (m, 4H).

Step E. To a solution of [5-(4-cyclopropyl-[1,4]diazepane-1-carbonyl)-2-(3,4-dichloro-phenoxy)-benzyl]-methyl-carbamic acid tert-butyl ester in DCM (1 mL) was added TFA (2 mL). After 30 min, the mixture was concentrated and the residue was purified by FCC to give the desired product (184 mg, 75% over 2 steps). MS (ESI): mass calcd. for C₂₃H₂₇Cl₂N₃O₂, 447.15; m/z found, 448.2 [M+H]⁺. ¹H NMR (CDCl₃): 7.53-7.48 (m, 1H), 7.39 (d, J=8.8, 1H), 7.32-7.28 (m, 1H), 7.08 (d, J=2.7, 1H), 6.89 (d, J=8.2, 1H), 6.85 (dd, J=8.8, 2.7, 1H), 3.79 (br s, 2H), 3.78-3.72 (m, 2H), 3.53-3.49 (m, 2H), 3.00-2.94 (m, 1H), 2.89-2.84 (m, 1H), 2.82-2.76 (m, 1H), 2.45 (s, 3H), 1.99-1.74 (m, 3H), 0.53-0.34 (m, 4H).

Compounds in Examples 2-39 were prepared by a sequence similar to that described in Example 1.

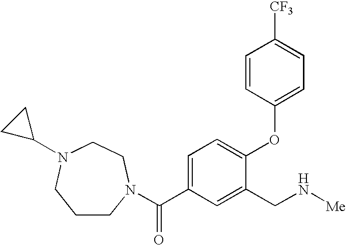

### Example 2

### (4-Cyclopropyl-[1,4]diazepan-1-yl)-[3-methylaminomethyl-4-(4-trifluoromethyl-phenoxy)-phenyl]-methanone

MS (ESI): mass calcd. for C₂₄H₂₈F₃N₃O₂, 447.21; m/z found, 448.4 [M+H]⁺. ¹H NMR (CDCl₃): 7.59 (d, J=8.5, 2H), 7.54-7.51 (m, 1H), 7.33-7.29 (m, 1H), 7.03 (d, J=8.4, 2H), 6.94 (d, J=8.3, 1H), 3.78-3.74 (m, 4H), 3.53-3.47 (m, 2H), 2.99-2.95 (m, 1H), 2.89-2.84 (m, 1H), 2.82-2.77 (m, 2H), 2.44 (s, 3H), 1.99-1.75 (m, 3H), 1.65 (br s, 1H), 0.52-0.35 (m, 4H).

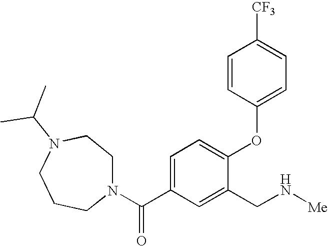

### Example 3

### (4-Isopropyl-[1,4]diazepan-1-yl)-[3-methylaminomethyl-4-(4-trifluoromethyl-phenoxy)-phenyl]-methanone

MS (ESI): mass calcd. for C₂₄H₃₀F₃N₃O₂, 449.23; m/z found, 450.4 [M+H]⁺. ¹H NMR (CDCl₃): 7.58 (d, J=8.7, 2H), 7.53-7.51 (m, 1H), 7.33-7.30 (m, 1H), 7.02 (d, J=8.6, 2H), 6.93 (d, J=8.3, 1H), 3.78-3.74 (m, 4H), 3.50-3.46 (m, 2H), 3.00-2.87 (m, 1H), 2.83-2.79 (m, 1H), 2.71-2.67 (m, 1H), 2.65-2.59 (m, 2H), 2.42 (s, 3H), 1.95-1.89 (m, 1H), 1.80-1.75 (m, 1H), 1.04 (d, J=6.6, 3H), 0.99 (d, J=6.5, 3H).

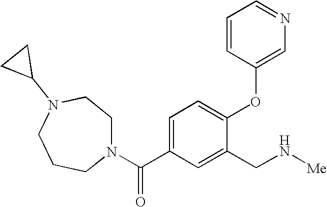

### Example 4

### (4-Cyclopropyl-[1,4]diazepan-1-yl)-[3-methylaminomethyl-4-(pyridin-3-yloxy)-phenyl]-methanone

MS (ESI): mass calcd. for C₂₂H₂₈N₄O₂, 380.22; m/z found, 381.4 [M+H]⁺. ¹H NMR (MeOD): 8.84 (s, 1H), 8.71 (d, J=4.5, 1H), 8.29 (dd, J=8.7, 1.7, 1H), 8.06-8.02 (m, 1H), 7.84-7.76 (m, 1H), 7.68-7.61 (m, 1H), 7.20 (d, J=8.5, 1H), 4.41 (s, 2H), 3.93-3.57 (m, 8H), 3.02-2.96 (m, 1H), 2.84 (s, 3H), 2.35-2.20 (m, 2H), 1.16-1.08 (m, 2H), 1.03-0.95 (m, 2H).

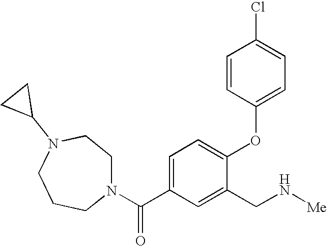

### Example 5

### [4-(4-Chloro-phenoxy)-3-methylaminomethyl-phenyl]-(4-cyclopropyl-[1,4]diazepan-1-yl)-methanone

MS (ESI): mass calcd. for C₂₃H₂₈ClN₃O₂, 413.19; m/z found, 414.3 [M+H]⁺. ¹H NMR (CDCl₃): 7.49-7.46 (m, 1H), 7.32-7.29 (m, 2H), 7.28-7.24 (m, 1H), 6.94-6.90 (m, 2H), 6.83 (d, J=8.3, 1H), 3.82 (s, 2H), 3.77-3.72 (m, 2H), 3.52-3.46 (m, 2H), 2.98-2.94 (m, 1H), 2.88-2.83 (m, 1H), 2.81-2.76 (m, 2H), 2.46 (s, 3H), 1.97-1.70 (m, 4H), 0.53-0.36 (m, 4H).

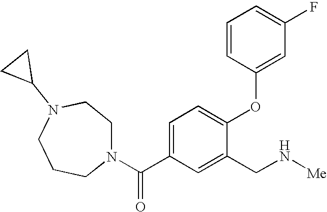

### Example 6

### (4-Cyclopropyl-[1,4]diazepan-1-yl)-[4-(3-fluoro-phenoxy)-3-methylaminomethyl-phenyl]-methanone

MS (ESI): mass calcd. for C₂₃H₂₈FN₃O₂, 397.22; m/z found, 398.4 [M+H]⁺. ¹H NMR (CDCl₃): 7.51-7.43 (m, 1H), 7.32-7.26 (m, 2H), 6.91 (d, J=8.3, 1H), 6.82 (tdd, J=8.3, 2.4, 0.84, 1H), 6.75 (dd, J=8.2, 2.2, 1H), 6.69 (dt, J=10.1, 2.3, 1H), 3.80 (s, 2H), 3.78-3.73 (m, 2H), 3.53-3.47 (m, 2H), 2.99-2.94 (m, 1H), 2.88-2.84 (m, 1H), 2.82-2.77 (m, 2H), 2.44 (s, 3H), 1.98-1.74 (m, 4H), 0.52-0.36 (m, 4H).

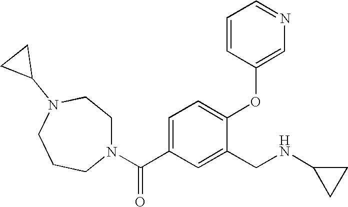

### Example 7

### [3-Cyclopropylaminomethyl-4-(pyridin-3-yloxy)-phenyl]-(4-cyclopropyl-[1,4]diazepan-1-yl)-methanone

MS (ESI): mass calcd. for C₂₄H₃₀N₄O₂, 406.24; m/z found, 407.4 [M+H]⁺. ¹H NMR (MeOD): 8.87 (s, 1H), 8.74-8.71 (m, 1H), 8.36-8.33 (m, 1H), 8.10-8.06 (m, 1H), 7.86-7.78 (m, 1H), 7.68-7.61 (m, 1H), 7.22 (d, J=8.5, 1H), 4.52 (s, 2H), 3.94-3.52 (m, 8H), 3.02-2.94 (m, 1H), 2.89-2.94 (m, 1H), 2.34-2.20 (m, 2H), 1.16-1.08 (m, 2H), 1.03-0.90 (m, 6H).

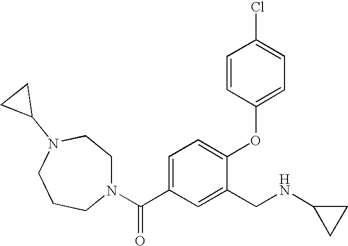

### Example 8

### [4-(4-Chloro-phenoxy)-3-cyclopropylaminomethyl-phenyl]-(4-cyclopropyl-[1,4]diazepan-1-yl)-methanone

MS (ESI): mass calcd. for C₂₅H₃₀ClN₃O₂, 439.20; m/z found, 440.4 [M+H]⁺. ¹H NMR (CDCl₃): 7.48-7.44 (m, 1H), 7.31 (d, J=9.0, 2H), 7.27-7.23 (m, 1H), 6.93 (d, J=9.0, 2H), 6.84 (d, J=8.3, 1H), 3.89 (s, 2H), 3.78-3.72 (m, 2H), 3.52-3.45 (m, 2H), 3.02-2.95 (m, 1H), 2.89-2.76 (m, 3H), 2.15-2.08 (m, 1H), 1.98-1.76 (m, 4H), 0.54-0.35 (m, 8H).

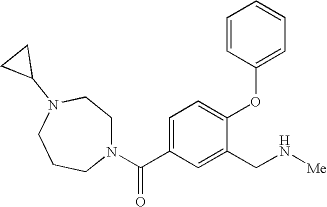

### Example 9

### (4-Cyclopropyl-[1,4]diazepan-1-yl)-(3-methylaminomethyl-4-phenoxy-phenyl)-methanone

MS (ESI): mass calcd. for C₂₃H₂₉N₃O₂, 379.23; m/z found, 380.4 [M+H]⁺. ¹H NMR (CDCl₃): 7.49-7.45 (m, 1H), 7.38-7.32 (m, 2H), 7.28-7.22 (m, 1H), 7.16-7.11 (m, 1H), 7.04-6.96 (m, 2H), 6.84 (d, J=8.2, 1H), 3.85 (br s, 2H), 3.78-3.71 (m, 2H), 3.53-3.40 (m, 2H), 3.00-2.92 (m, 1H), 2.89-2.82 (m, 1H), 2.81-2.74 (m, 2H), 2.50 (s, 3H), 1.98-1.72 (m 3H), 0.53-0.33 (m, 4H).

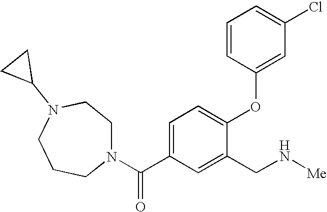

### Example 10

### [4-(3-Chloro-phenoxy)-3-methylaminomethyl-phenyl]-(4-cyclopropyl-[1,4]diazepan-1-yl)-methanone

MS (ESI): mass calcd. for C₂₃H₂₈ClN₃O₂, 413.19; m/z found, 414.3 [M+H]⁺. ¹H NMR (CDCl₃): 7.51-7.48 (m, 1H), 7.31-7.23 (m, 2H), 7.11-7.07 (m, 1H), 6.97 (t, J=2.1, 1H), 6.89 (d, J=8.3, 1H), 6.86 (ddd, J=8.3, 2.4, 0.92, 1H), 3.78 (s, 2H), 3.77-3.73 (m, 2H), 3.53-3.47 (m, 2H), 2.98-2.94 (m, 1H), 2.88-2.84 (m, 1H), 2.82-2.77 (m, 2H), 2.44 (s, 3H), 1.98-1.76 (m, 3H), 1.61 (br s, 1H), 0.52-0.35 (m, 4H).

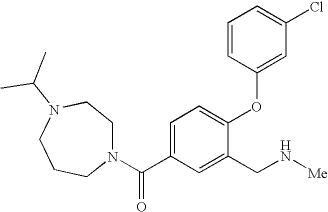

### Example 11

### [4-(3-Chloro-phenoxy)-3-methylaminomethyl-phenyl]-(4-isopropyl-[1,4]diazepan-1-yl)-methanone

MS (ESI): mass calcd. for C₂₃H₃₀ClN₃O₂, 415.20; m/z found, 416.7 [M+H]⁺. ¹H NMR (CDCl₃): 7.50-7.47 (m, 1H), 7.31-7.27 (m, 1H), 7.24 (d, J=8.2, 1H), 7.10-7.07 (m, 1H), 6.97-6.95 (m, 1H), 6.89 (d, J=8.3, 1H), 6.87-6.83 (m, 1H), 3.78-3.73 (m, 4H), 3.51-3.46 (m, 2H), 2.99-2.84 (m, 1H), 2.81-2.77 (m, 1H), 2.71-2.66 (m, 1H), 2.63-2.58 (m, 2H), 2.43 (s, 3H), 1.96-1.83 (m, 1H), 1.79-1.73 (m, 1H), 1.03 (d, J=6.6, 3H), 0.98 (d, J=6.6, 3H).

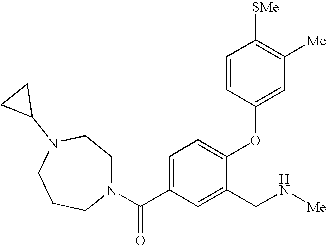

### Example 12

### (4-Cyclopropyl-[1,4]diazepan-1-yl)-[3-methylaminomethyl-4-(3-methyl-4-methylsulfanyl-phenoxy)-phenyl]-methanone

MS (ESI): mass calcd. for C₂₅H₃₃N₃O₂S, 439.23; m/z found, 440.4 [M+H]⁺. ¹H NMR (CDCl₃): 7.47-7.43 (m, 1H), 7.27-7.22 (m, 1H), 7.18-7.15 (m, 1H), 6.84-6.80 (m, 3H), 3.82 (s, 2H), 3.77-3.73 (m, 2H), 3.54-3.46 (m, 2H), 2.99-2.94 (m, 1H), 2.88-2.83 (m, 1H), 2.81-2.76 (m, 2H), 2.47-2.43 (m, 6H), 2.34 (s, 3H), 1.97-1.75 (m, 3H), 1.68 (br s, 1H), 0.52-0.35 (m, 4H).

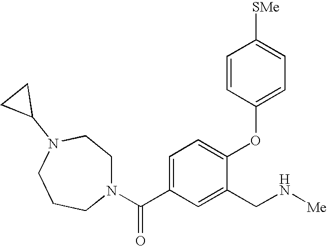

### Example 13

### (4-Cyclopropyl-[1,4]diazepan-1-yl)-[3-methylaminomethyl-4-(4-methylsulfanyl-phenoxy)-phenyl]-methanone

MS (ESI): mass calcd. for C₂₄H₃₁N₃O₂S, 425.21; m/z found, 426.4 [M+H]⁺. ¹H NMR (CDCl₃): 7.50 (d, J=8.6, 1H), 7.30-7.24 (m, 3H), 6.96 (d, J=8.7, 2H), 6.80 (d, J=8.3, 1H), 3.94 (s, 2H), 3.75-3.70 (m, 2H), 3.52-3.46 (m, 2H), 2.98-2.93 (m, 1H), 2.87-2.83 (m, 1H), 2.80-2.75 (m, 2H), 2.51 (s, 3H), 2.49 (s, 3H), 1.96-1.76 (m, 4H), 0.52-0.36 (m, 4H).

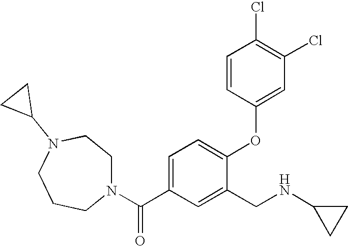

### Example 14

### [3-Cyclopropylaminomethyl-4-(3,4-dichloro-phenoxy)-phenyl]-(4-cyclopropyl-[1,4]diazepan-1-yl)-methanone

MS (ESI): mass calcd. for C₂₅H₂₉Cl₂N₃O₂, 473.16; m/z found, 474.3 [M+H]⁺. ¹H NMR (CDCl₃): 7.51-7.45 (m, 1H), 7.39 (d, J=8.8, 1H), 7.31-7.26 (m, 1H), 7.07 (d, J=2.8, 1H), 6.89 (d, J=8.3, 1H), 6.83 (dd, J=8.8, 2.8, 1H), 3.85 (s, 2H), 3.78-3.73 (m, 2H), 3.52-3.45 (m, 2H), 2.99-2.94 (m, 1H), 2.89-2.84 (m, 1H), 2.82-2.76 (m, 2H), 2.13-2.07 (m, 1H), 1.98-1.76 (m, 3H), 0.52-0.33 (m, 8H).

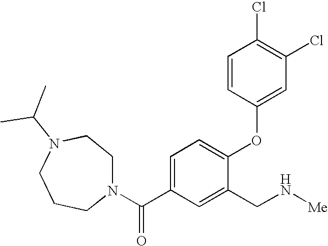

### Example 15

### [4-(3,4-Dichloro-phenoxy)-3-methylaminomethyl-phenyl]-(4-isopropyl-[1,4]diazepan-1-yl)-methanone

MS (ESI): mass calcd. for C₂₃H₂₉Cl₂N₃O₂, 449.16; m/z found, 450.3 [M+H]⁺. ¹H NMR (CDCl₃): 7.54-7.48 (m, 1H), 7.39 (d, J=8.8, 1H), 7.34-7.28 (m, 1H), 7.07 (d, J=2.7, 1H), 6.89 (d, J=8.2, 1H), 6.83 (dd, J=8.8, 2.7, 1H), 3.82-3.72 (m, 4H), 3.54-3.44 (m, 2H), 3.06-2.74 (m, 2H), 2.74-2.56 (m, 3H), 2.44 (s, 3H), 1.99-1.88 (m, 1H), 1.87-1.54 (m, 1H), 1.06 (d, J=6.6, 3H), 0.99 (d, J=6.6, 3H).

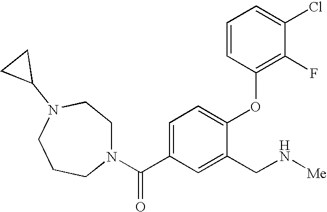

### Example 16

### [4-(3-Chloro-2-fluoro-phenoxy)-3-methylaminomethyl-phenyl]-(4-cyclopropyl-[1,4]diazepan-1-yl)-methanone

MS (ESI): mass calcd. for C₂₃H₂₇ClFN₃O₂, 431.18; m/z found, 432.3 [M+H]⁺. ¹H NMR (CDCl₃): 7.50-7.46 (m, 1H), 7.26-7.19 (m, 2H), 7.06 (td, J=8.2, 1.8, 1H), 6.95-6.90 (m, 1H), 6.75 (d, J=8.3, 1H), 3.87 (s, 2H), 3.77-3.72 (m, 2H), 3.52-3.45 (m, 2H), 2.98-2.94 (m, 1H), 2.88-2.83 (m, 1H), 2.80-2.76 (m, 2H), 2.47 (s, 3H), 1.99-1.74 (m, 3H), 0.52-0.35 (m, 4H).

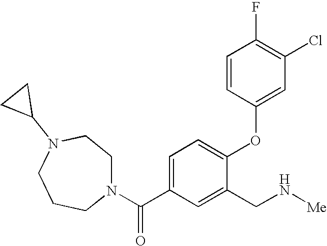

### Example 17

### [4-(3-Chloro-4-fluoro-phenoxy)-3-methylaminomethyl-phenyl]-(4-cyclopropyl-[1,4]diazepan-1-yl)-methanone

MS (ESI): mass calcd. for C₂₃H₂₇ClFN₃O₂, 431.18; m/z found, 432.3 [M+H]⁺. ¹H NMR (CDCl₃): 7.51-7.47 (m, 1H), 7.30-7.26 (m, 1H), 7.13 (t, J=8.8, 1H), 7.06-7.03 (m, 1H), 6.90-6.85 (m, 1H), 6.83 (d, J=8.4, 1H), 3.81 (s, 2H), 3.77-3.72 (m, 2H), 3.52-3.46 (m, 2H), 2.98-2.95 (m, 1H), 2.88-2.84 (m, 1H), 2.81-2.77 (m, 2H), 2.46 (s, 3H), 1.97-1.75 (m, 4H), 0.53-0.35 (m, 4H).

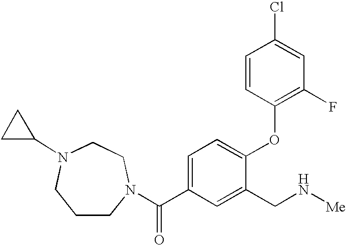

### Example 18

### [4-(4-Chloro-2-fluoro-phenoxy)-3-methylaminomethyl-phenyl]-(4-cyclopropyl-[1,4]diazepan-1-yl)-methanone

MS (ESI): mass calcd. for C₂₃H₂₇ClFN₃O₂, 431.18; m/z found, 432.3 [M+H]⁺. ¹H NMR (CDCl₃): 7.48-7.44 (m, 1H), 7.26-7.21 (m, 2H), 7.14-7.10 (m, 1H), 7.00 (t, J=8.6, 1H), 6.71 (d, J=8.4, 1H), 3.87 (s, 2H), 3.77-3.72 (m, 2H), 3.51-3.45 (m, 2H), 2.98-2.94 (m, 1H), 2.87-2.83 (m, 1H), 2.81-2.76 (m, 2H), 2.46 (s, 3H), 1.98-1.74 (m, 4H), 0.52-0.35 (m, 4H).

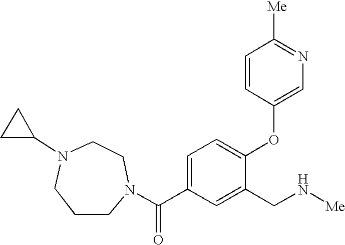

### Example 19

### (4-Cyclopropyl-[1,4]diazepan-1-yl)-[3-methylaminomethyl-4-(6-methyl-pyridin-3-yloxy)-phenyl]-methanone

MS (ESI): mass calcd. for C₂₃H₃₀N₄O₂, 394.24; m/z found, 395.4 [M+H]⁺. ¹H NMR (CDCl₃): 8.29 (d, J=2.4, 1H), 7.50-7.46 (m, 1H), 7.27-7.23 (m, 1H), 7.19 (dd, J=8.5, 2.8, 1H), 7.14 (d, J=8.4, 1H), 6.80 (d, J=8.3, 1H), 3.83 (s, 2H), 3.77-3.72 (m, 2H), 3.52-3.45 (m, 2H), 2.98-2.94 (m, 1H), 2.88-2.83 (m, 1H), 2.81-2.76 (m, 2H), 2.56 (s, 3H), 2.46 (s, 3H), 1.99-1.74 (m, 3H), 0.53-0.34 (m, 4H).

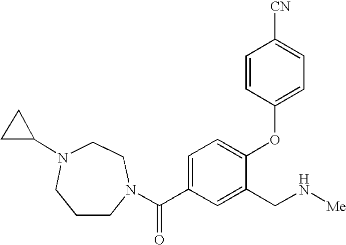

### Example 20

### 4-[4-(4-Cyclopropyl-[1,4]diazepane-1-carbonyl)-2-methylaminomethyl-phenoxy]-benzonitrile

MS (ESI): mass calcd. for C₂₄H₂₈N₄O₂, 404.22; m/z found, 405.4 [M+H]⁺. ¹H NMR (MeOD): 7.64 (br s, 1H), 7.57-7.56 (m, 2H), 7.52-7.48 (m, 2H), 7.43-7.41 (m, 1H), 6.88 (d, J=8.4, 1H), 4.28 (s, 2H), 3.83-3.39 (br m, 7H), 2.89 (br s, 1H), 2.72 (s, 3H), 2.24-2.09 (br m, 2H), 1.04-0.86 (br m, 4H).

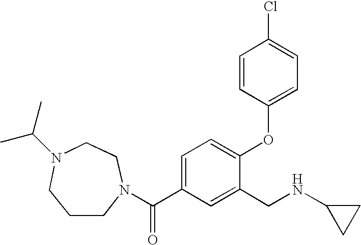

### Example 21

### [4-(4-Chloro-phenoxy)-3-cyclopropylaminomethyl-phenyl]-(4-isopropyl-[1,4]diazepan-1-yl)-methanone

MS (ESI): mass calcd. for C₂₅H₃₂ClN₃O₂, 441.22; m/z found, 442.3 [M+H]⁺. ¹H NMR (CDCl₃): 7.48-7.46 (m, 1H), 7.30 (d, J=9.0, 2H), 7.28-7.24 (m, 1H), 6.94-6.90 (m, 2H), 6.84 (d, J=8.3, 1H), 3.87 (s, 2H), 3.77-3.73 (m, 2H), 3.50-3.45 (m, 2H), 2.99-2.85 (m, 1H), 2.82-2.77 (m, 1H), 2.71-2.66 (m, 1H), 2.64-2.53 (m, 2H), 2.14-2.08 (m, 1H), 1.96-1.71 (m, 2H), 1.05-0.97 (m, 6H), 0.44-0.33 (m, 4H).

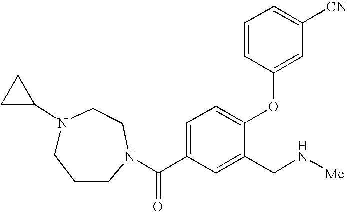

### Example 22

### 3-[4-(4-Cyclopropyl-[1,4]diazepane-1-carbonyl)-2-methylaminomethyl-phenoxy]-benzonitrile

MS (ESI): mass calcd. for C₂₄H₂₈N₄O₂, 404.22; m/z found, 405.4 [M+H]⁺. ¹H NMR (MeOD): 7.84 (d, J=8.9, 2H), 7.78-7.69 (m, 1H), 7.65-7.58 (m, 1H), 7.32 (d, J=8.9, 2H), 7.09 (d, J=8.5, 1H), 4.35 (s, 2H), 3.92-3.56 (m, 8H), 3.02-2.95 (m, 1H), 2.81 (s, 3H), 2.33-2.20 (m, 2H), 1.13-0.97 (m, 4H).

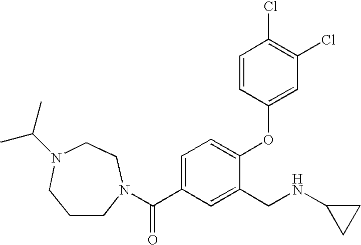

### Example 23

### [3-Cyclopropylaminomethyl-4-(3,4-dichloro-phenoxy)-phenyl]-(4-isopropyl-[1,4]diazepan-1-yl)-methanone

MS (ESI): mass calcd. for C₂₅H₃₁Cl₂N₃O₂, 475.18; m/z found, 476.3 [M+H]⁺. ¹H NMR (CDCl₃): 7.51-7.48 (m, 1H), 7.39 (d, J=8.8, 1H), 7.32-7.28 (m, 1H), 7.07 (d, J=2.8, 1H), 6.89 (d, J=8.3, 1H), 6.83 (dd, J=8.8, 2.8, 1H), 3.85 (s, 2H), 3.78-3.73 (m, 2H), 3.50-3.45 (m, 2H), 2.99-2.85 (m, 1H), 2.82-2.77 (m, 1H), 2.71-2.66 (m, 1H), 2.65-2.59 (m, 2H), 2.13-2.07 (m, 1H), 1.96-1.89 (m, 1H), 1.85-1.71 (m, 2H), 1.04 (d, J=6.6, 3H), 0.99 (d, J=6.6, 3H), 0.44-0.32 (m, 4H).

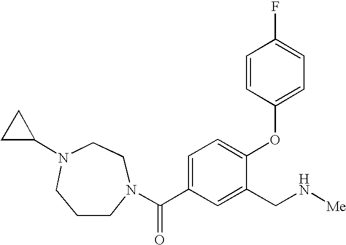

### Example 24

### (4-Cyclopropyl-[1,4]diazepan-1-yl)-[4-(4-fluoro-phenoxy)-3-methylaminomethyl-phenyl]-methanone

MS (ESI): mass calcd. for C₂₃H₂₈FN₃O₂, 397.22; m/z found, 398.4 [M+H]⁺. ¹H NMR (CDCl₃): 7.47-7.44 (m, 1H), 7.26-7.21 (m, 1H), 7.07-7.02 (m, 2H), 6.98-6.94 (m, 2H), 6.77 (d, J=8.3, 1H), 3.83 (s, 2H), 3.77-3.72 (m, 2H), 3.52-3.46 (m, 2H), 2.98-2.94 (m, 1H), 2.88-2.83 (m, 1H), 2.81-2.76 (m, 2H), 2.46 (s, 3H), 1.98-1.73 (m, 3H), 0.51-0.35 (m, 4H).

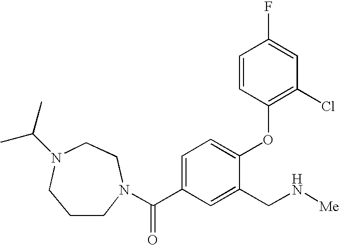

### Example 25

### [4-(2-Chloro-4-fluoro-phenoxy)-3-methylaminomethyl-phenyl]-(4-isopropyl-[1,4]diazepan-1-yl)-methanone

MS (ESI): mass calcd. for C₂₃H₂₉ClFN₃O₂, 433.19; m/z found, 434.8 [M+H]⁺. ¹H NMR (CDCl₃): 7.47-7.44 (m, 1H), 7.26-7.21 (m, 2H), 7.01-6.98 (m, 2H), 6.60 (d, J=8.4, 1H), 3.87 (s, 2H), 3.77-3.72 (m, 2H), 3.50-3.44 (m, 2H), 2.98-2.84 (m, 1H), 2.81-2.76 (m, 1H), 2.71-2.66 (m, 1H), 2.63-2.57 (m, 2H), 2.46 (s, 3H), 1.95-1.87 (m, 1H), 1.79-1.71 (m, 1H), 1.03 (d, J=6.6, 3H), 0.98 (d, J=6.5, 3H).

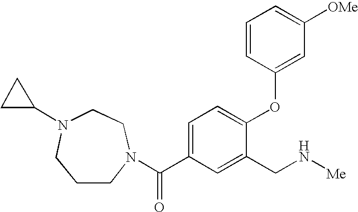

### Example 26

### (4-Cyclopropyl-[1,4]diazepan-1-yl)-[4-(3-methoxy-phenoxy)-3-methylaminomethyl-phenyl]-methanone

MS (ESI): mass calcd. for C₂₄H₃₁N₃O₃, 409.24; m/z found, 410.4 [M+H]⁺. ¹H NMR (CDCl₃): 7.47-7.44 (m, 1H), 7.27-7.21 (m, 2H), 6.89 (d, J=8.3, 1H), 6.69-6.65 (m, 1H), 6.56-6.53 (m, 2H), 3.81 (s, 2H), 3.79 (s, 3H), 3.77-3.73 (m, 2H), 3.54-3.47 (m, 2H), 2.98-2.94 (m, 1H), 2.88-2.83 (m, 1H), 2.81-2.76 (m, 2H), 2.44 (s, 3H), 1.98-1.74 (m, 3H), 0.53-0.35 (m, 4H).

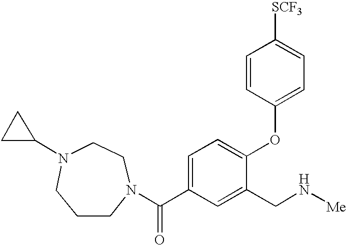

### Example 27

### (4-Cyclopropyl-[1,4]diazepan-1-yl)-[3-methylaminomethyl-4-(4-trifluoromethylsulfanyl-phenoxy)-phenyl]-methanone

MS (ESI): mass calcd. for C₂₄H₂₈F₃N₃O₂S, 479.19; m/z found, 480.3 [M+H]⁺. ¹H NMR (MeOD): 7.79 (d, J=8.7, 2H), 7.75-7.66 (m, 1H), 7.63-7.55 (m, 1H), 7.28 (d, J=8.8, 2H), 7.04 (d, J=8.5, 1H), 4.35 (s, 2H), 3.89-3.56 (m, 8H), 3.02-2.96 (m, 1H), 2.80 (s, 3H), 2.32-2.17 (m, 2H), 1.10-0.96 (m, 4H).

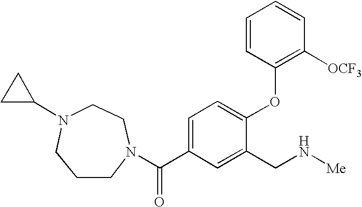

### Example 28

### (4-Cyclopropyl-[1,4]diazepan-1-yl)-[3-methylaminomethyl-4-(2-trifluoromethoxy-phenoxy)-phenyl]-methanone

MS (ESI): mass calcd. for C₂₄H₂₈F₃N₃O₃, 463.21; m/z found, 464.3 [M+H]⁺. ¹H NMR (MeOD): 7.76-7.78 (m, 1H), 7.60-7.46 (m, 3H), 7.46-7.40 (m, 1H), 7.39-7.35 (m, 1H), 6.84 (d, J=8.6, 1H), 4.43 (s, 2H), 3.89-3.83 (m, 2H), 3.69-3.51 (m, 5H), 3.48-3.44 (m, 1H), 3.02-2.95 (m, 1H), 2.82 (s, 3H), 2.32-2.26 (m, 1H), 2.24-2.18 (m, 1H), 1.12-1.05 (m, 2H), 1.04-0.98 (m, 3H).

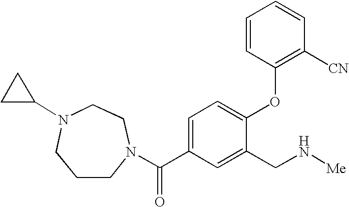

### Example 29

### 2-[4-(4-Cyclopropyl-[1,4]diazepane-1-carbonyl)-2-methylaminomethyl-phenoxy]-benzonitrile

MS (ESI): mass calcd. for C₂₄H₂₈N₄O₂, 404.22; m/z found, 405.4 [M+H]⁺. ¹H NMR (MeOD): 7.89 (dd, J=7.8, 1.5, 1H), 7.82-7.21 (m, 2H), 7.65-7.57 (m, 1H), 7.50-7.46 (m, 1H), 7.35 (d, J=8.3, 1H), 6.97 (d, J=8.5, 1H), 4.45 (s, 2H), 3.92-3.56 (m, 8H), 3.03-2.91 (m, 1H), 2.84 (s, 3H), 2.33-2.18 (m, 2H), 1.13-0.97 (m, 4H).

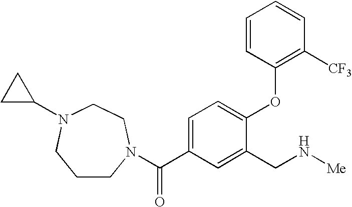

### Example 30

### (4-Cyclopropyl-[1,4]diazepan-1-yl)-[3-methylaminomethyl-4-(2-trifluoromethyl-phenoxy)-phenyl]-methanone

MS (ESI): mass calcd. for C₂₄H₂₈F₃N₃O₂, 447.21; m/z found, 448.4 [M+H]⁺. ¹H NMR (MeOD): 7.86 (d, J=7.8, 1H), 7.77-7.72 (m, 2H), 7.62-7.58 (m, 1H), 7.49 (t, J=7.6, 1H), 7.30 (d, J=8.3, 1H), 6.91 (d, J=8.5, 1H), 4.41 (s, 2H), 3.92-3.44 (m, 9H), 3.02-2.93 (m, 1H), 2.81 (s, 3H), 2.32-2.18 (m, 2H), 1.12-0.96 (m, 4H).

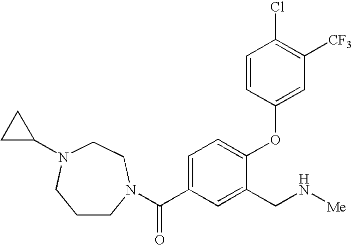

### Example 31

### [4-(4-Chloro-3-trifluoromethyl-phenoxy)-3-methylaminomethyl-phenyl]-(4-cyclopropyl-[1,4]diazepan-1-yl)-methanone

MS (ESI): mass calcd. for C₂₄H₂₇ClF₃N₃O₂, 481.17; m/z found, 482.8 [M+H]⁺. ¹H NMR (CDCl₃): 7.54-7.50 (m, 1H), 7.44 (d, J=2.8, 1H), 7.33 (d, J=2.8, 1H), 7.32-7.28 (m, 1H), 7.03 (dd, J=8.8, 2.9, 1H), 6.88 (d, J=8.3, 1H), 3.79-3.73 (m, 4H), 3.52-3.46 (m, 2H), 2.98-2.94 (m, 1H), 2.89-2.84 (m, 1H), 2.82-2.77 (m, 2H), 2.44 (s, 3H), 1.98-1.75 (m, 3H), 0.53-0.36 (m, 4H).

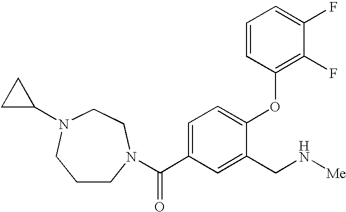

### Example 32

### (4-Cyclopropyl-[1,4]diazepan-1-yl)-[4-(2,3-difluoro-phenoxy)-3-methylaminomethyl-phenyl]-methanone

MS (ESI): mass calcd. for C₂₃H₂₇F₂N₃O₂, 415.21; m/z found, 416.8 [M+H]⁺. ¹H NMR (CDCl₃): 7.50-7.46 (m, 1H), 7.27-7.22 (m, 1H), 7.06-6.95 (m, 2H), 6.80-6.75 (m, 2H), 3.86 (s, 2H), 3.77-3.72 (m, 2H), 3.51-3.44 (m, 2H), 2.98-2.93 (m, 1H), 2.87-2.83 (m, 1H), 2.80-2.75 (m, 2H), 2.46 (s, 3H), 1.97-1.74 (m, 3H), 0.51-0.35 (m, 4H).

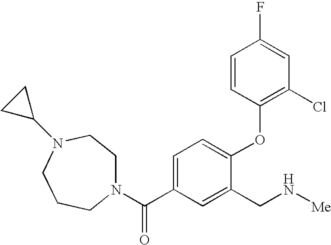

### Example 33

### [4-(2-Chloro-4-fluoro-phenoxy)-3-methylaminomethyl-phenyl]-(4-cyclopropyl-[1,4]diazepan-1-yl)-methanone

MS (ESI): mass calcd. for C₂₃H₂₇ClFN₃O₂, 431.18; m/z found, 432.7 [M+H]⁺. ¹H NMR (CDCl₃): 7.47-7.44 (m, 1H), 7.25-7.19 (m, 2H), 7.01-6.98 (m, 2H), 6.60 (d, J=8.3, 1H), 3.88 (s, 2H), 3.76-3.71 (m, 2H), 3.51-3.45 (m, 2H), 2.98-2.91 (m, 1H), 2.87-2.73 (m, 3H), 2.47 (s, 3H), 1.96-1.74 (m, 3H), 0.52-0.35 (m, 4H).

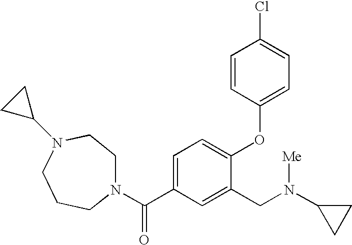

### Example 34

### {4-(4-Chloro-phenoxy)-3-[(cyclopropyl-methyl-amino)-methyl]-phenyl}-(4-cyclopropyl-[1,4]diazepan-1-yl)-methanone

MS (ESI): mass calcd. for C₂₆H₃₂ClN₃O₂, 453.22; m/z found, 454.8 [M+H]⁺. ¹H NMR (CDCl₃): 7.47 (br s, 1H), 7.30-7.24 (m, 3H), 6.90-6.84 (m, 3H), 3.78-3.72 (m, 2H), 3.71 (s, 2H), 3.51-3.44 (m, 2H), 2.99-2.94 (m, 1H), 2.88-2.83 (m, 1H), 2.80-2.76 (m, 2H), 2.27 (s, 3H), 1.98-1.72 (m, 4H), 0.54-0.35 (m, 8H).

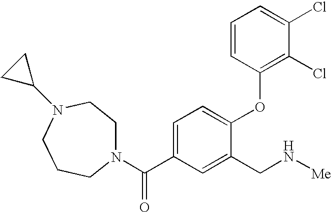

### Example 35

### (4-Cyclopropyl-[1,4]diazepan-1-yl)-[4-(2,3-dichloro-phenoxy)-3-methylaminomethyl-phenyl]-methanone

MS (ESI): mass calcd. for C₂₃H₂₇Cl₂N₃O₂, 447.15; m/z found, 448.8 [M+H]⁺. ¹H NMR (CDCl₃): 7.50-7.47 (m, 1H), 7.30-7.22 (m, 1H), 7.17 (t, J=8.2, 1H), 6.85 (d, J=8.3, 1H), 6.73 (d, J=8.3, 1H), 3.83 (s, 2H), 3.77-3.72 (m, 2H), 3.53-3.45 (m, 2H), 2.99-2.94 (m, 1H), 2.88-2.76 (m, 3H), 2.45 (s, 3H), 1.97-1.64 (m, 4H), 0.52-0.34 (m, 4H).

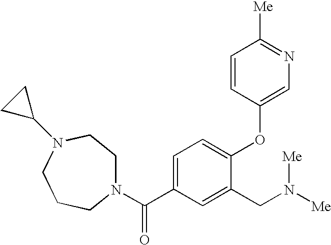

### Example 36

### (4-Cyclopropyl-[1,4]diazepan-1-yl)-[3-dimethylaminomethyl-4-(6-methyl-pyridin-3-yloxy)-phenyl]-methanone

MS (ESI): mass calcd. for C₂₄H₃₂N₄O₂, 408.25; m/z found, 409.9 [M+H]⁺. ¹H NMR (CDCl₃): 8.26-8.23 (m, 1H), 7.57-7.51 (m, 1H), 7.27-7.22 (m, 1H), 7.16-7.07 (m, 2H), 6.82 (d, J=8.3, 1H), 3.76-3.69 (m, 2H), 3.55-3.44 (m, 4H), 2.97-2.92 (m, 1H), 2.87-2.74 (m, 3H), 2.52 (s, 3H), 2.25 (s, 6H), 1.96-1.71 (m, 3H), 0.51-0.33 (m, 4H).

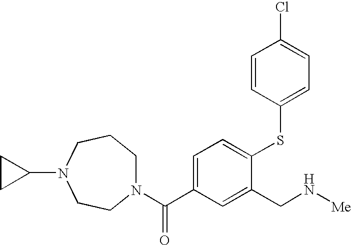

### Example 37

### [4-(4-Chloro-phenylsulfanyl)-3-methylaminomethyl-phenyl]-(4-cyclopropyl-[1,4]diazepan-1-yl)-methanone

MS (ESI): mass calcd. for C₂₃H₂₈ClN₃OS, 429.16; m/z found, 430.3 [M+H]⁺. ¹H NMR (CDCl₃): 7.44 (d, J=6.0, 1H), 7.29-7.24 (m, 2H), 7.22-7.16 (m, 4H), 3.84 (s, 2H), 3.74-3.70 (m, 2H), 3.45-3.40 (m, 2H), 2.95-2.91 (m, 1H), 2.85-2.81 (m, 1H), 2.78-2.73 (m, 2H), 2.42 (s, 3H), 1.94-1.88 (m, 1H), 1.88-1.77 (m, 1H), 1.77-1.71 (m, 1H), 1.71-1.63 (m, 1H), 0.49-0.37 (m, 3H), 0.37-0.32 (m, 1H).

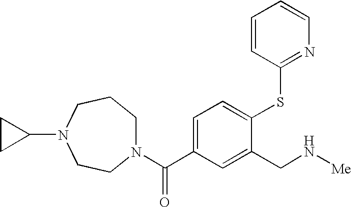

### Example 38

### (4-Cyclopropyl-[1,4]diazepan-1-yl)-[3-methylaminomethyl-4-(pyridin-2-ylsulfanyl)-phenyl]-methanone

MS (ESI): mass calculated for C₂₂H₂₈N₄OS, 396.20; m/z found, 397.8 [M+H]⁺. ¹H NMR (CDCl₃): 8.43-8.38 (m, 1H), 7.88-7.81 (m, 1H), 7.78-7.74 (m, 2H), 7.64 (d, J=7.8, 1H), 7.41 (dd, J=7.9, 1.6, 1H), 7.29-7.25 (m, 1H), 4.70-4.26 (m, 3H), 3.92-3.17 (m, 8H), 2.84 (br s, 3H), 2.76-1.90 (m, 3H), 1.32-1.20 (m, 2H), 0.94-0.83 (m, 2H).

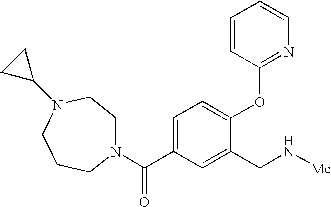

### Example 39

### (4-Cyclopropyl-[1,4]diazepan-1-yl)-[3-methylaminomethyl-4-(pyridin-2-yloxy)-phenyl]-methanone

MS (ESI): mass calcd. for C₂₂H₂₈N₄O₂, 380.22; m/z found, 381.4 [M+H]⁺. ¹H NMR (CDCl₃): 8.18 (dd, J=4.8, 1.4, 1H), 7.73-7.68 (m, 1H), 7.50 (d, J=4.2, 1H), 7.34-7.30 (m, 1H), 7.05 (d, J=8.3, 1H), 7.02 (ddd, J=7.2, 5.0, 0.9, 1H), 6.92 (d, J=8.3, 1H), 3.74 (br s, 4H), 3.54-3.50 (m, 2H), 2.97-2.94 (m, 1H), 2.88-2.83 (m, 1H), 2.80-2.76 (m, 2H), 2.39 (s, 3H), 1.93 (br s, 1H), 1.89-1.78 (m, 2H), 1.50-1.36 (m, 1H), 0.49-0.37 (m, 4H).

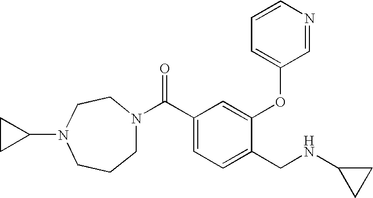

### Example 40

### [4-Cyclopropylaminomethyl-3-(pyridin-3-yloxy)-phenyl]-(4-cyclopropyl-[1,4]diazepan-1-yl)-methanone

Step A: 4-Bromo-2-(pyridin-3-yloxy)-benzaldehyde. To a solution of 4-bromo-2-fluoro-benzaldehyde (3.00 g, 14.8 mmol) in DMF (25 mL) were added K₂CO₃ (3.67 g, 26.6 mmol) and 3-hydroxypyridine (1.54 g, 28.8 mmol). The reaction was heated at 90° C. for 18 h then allowed to cool to rt. Water was added and the mixture was extracted with DCM. The combined organic layers were dried (MgSO₄) and concentrated. FCC purification (MeOH/DCM) provided the desired product (2.85 g, 71%). MS (ESI): mass calcd. for C₁₂H₈BrNO₂, 276.97; m/z found, 278.0, 280.0 [M+H]⁺. ¹H NMR (CDCl₃): 10.47 (s, 1H), 8.53 (dd, J=4.5, 1.3, 1H), 8.51 (d, J=2.7, 1H), 7.83 (d, J=8.4, 1H), 7.45-7.42 (m, 1H), 7.42-7.38 (m, 2H), 7.02-7.01 (m, 1H).

Step B: [4-Bromo-2-(pyridin-3-yloxy)-benzyl]-cyclopropyl-amine. To a mixture of 4-bromo-2-(pyridin-3-yloxy)-benzaldehyde (2.0 g, 7.19 mmol) in DCE (75 mL) were added cyclopropylamine (0.50 mL, 7.2 mmol), acetic acid (2.16 mL, 36.0 mmol), and NaBH(OAc)₃ (95%; 3.62 g, 18.0 mmol) portionwise. After 18 h, the mixture was diluted with 50 mL DCM and washed with 1 M NaOH (2×25 mL). The organic layer was dried (Na₂SO₄) and concentrated. FCC purification (EtOAc/DCM) gave the desired product (1.74 g, 76%). MS (ESI): mass calcd. for C₁₅H₁₅BrN₂O, 318.04; m/z found, 319.1, 321.1 [M+H]⁺. ¹H NMR (CDCl₃): 8.42-8.39 (m, 2H), 7.31-7.26 (m, 4H), 7.00-6.99 (m, 1H), 3.84 (s, 2H), 2.11-2.07 (m, 1H), 0.43-0.40 (m, 2H), 0.36-0.33 (m, 2H).

Step C: [4-Bromo-2-(pyridin-3-yloxy)-benzyl]-cyclopropyl-carbamic acid tert-butyl ester. To a solution [4-bromo-2-(pyridin-3-yloxy)-benzyl]-cyclopropyl-amine (1.67 g, 5.23 mmol) in DCM (50 mL) was added BOC anhydride (1.26 g, 5.75 mmol). The mixture was stirred at rt for 2 h and then concentrated. The crude material was carried forward without purification. MS (ESI): mass calcd. for C₂₀H₂₃BrN₂O₃, 418.09; m/z found, 419.1, 421.1 [M+H]⁺.

Step D: Cyclopropyl-[4-(4-cyclopropyl-[1,4]diazepane-1-carbonyl)-2-(pyridin-3-yloxy)-benzyl]-carbamic acid tert-butyl ester. To a solution of [4-bromo-2-(pyridin-3-yloxy)-benzyl]-cyclopropyl-carbamic acid tert-butyl ester (300 mg, 0.72 mmol) in THF (3 mL) were added DBU (0.33 g, 2.1 mmol), cyclopropylhomopiperazine (0.29 g, 2.1 mmol), Hermann's catalyst (27 mg, 0.03 mmol), t-BuPHBF₄⁺ (14 mg, 0.05 mmol), and Mo(CO)₆ (170 mg, 0.64 mmol). The mixture was heated in the microwave for 6 min at 125° C., cooled to rt, and then concentrated. FCC purification (EtOAc/MeOH/DCM) gave the desired product. MS (ESI): mass calcd. for C₂₉H₃₈N₄O, 506.64; m/z found, 507.2 [M+H]⁺.

Step E. To a solution of cyclopropyl-[4-(4-cyclopropyl-[1,4]diazepane-1-carbonyl)-2-(pyridin-3-yloxy)-benzyl]-carbamic acid tert-butyl ester (130 mg, 0.26 mmol) in DCM (3 mL) was added TFA (1 mL). After 18 h at rt, the mixture was concentrated. FCC purification (2 M N₃ in MeOH/DCM) gave the desired product (69.5 mg, 66%). MS (ES I): mass calcd. for C₂₄H₃₀N₄O₂, 406.24; m/z found, 407.2 [M+H]⁺. ¹H NMR (CDCl₃): 8.42-8.37 (m, 2H), 7.46 (d, J=7.7, 1H), 7.28-7.26 (m, 2H), 7.18-7.15 (m, 1H), 6.88 (d, J=1.2, 1H), 3.90 (s, 2H), 3.71-3.67 (m, 2H), 3.42-3.37 (m, 2H), 2.94-2.80 (m, 2H), 2.76-2.70 (m, 2H), 2.15-2.10 (m, 1H), 1.93-1.68 (m, 4H), 0.50-0.32 (m, 8H).

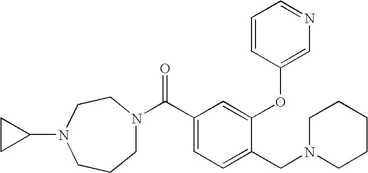

### Example 41

### (4-Cyclopropyl-[1,4]diazepan-1-yl)-[4-piperidin-1-ylmethyl-3-(pyridin-3-yloxy)-phenyl]-methanone

Step A: 3-(5-Bromo-2-piperidin-1-ylmethyl-phenoxy)-pyridine. The title compound was prepared in a similar manner as in Example 40, Step B (0.91 g, 85%). MS (ESI): mass calcd. for C₁₇H₁₉BrN₂O, 346.07; m/z found, 347.1, 349.1 [M+H]⁺. ¹H NMR (CDCl₃): 8.39-8.36 (m, 2H), 7.42 (d, J=8.2, 1H), 7.32 (dd, J=8.2, 1.9, 1H), 7.30-7.26 (m, 1H), 7.23-7.20 (m, 1H), 7.06 (d, J=1.9, 1H), 3.46 (s, 2H), 2.38 (br s, 4H), 1.55-1.48 (m, 4H), 1.44-1.37 (m, 2H).

Step B: (4-Cyclopropyl-[1,4]diazepan-1-yl)-[4-piperidin-1-ylmethyl-3-(pyridin-3-yloxy)-phenyl]-methanone. The title compound was prepared in a similar manner as in Example 40, Step D. MS (ESI): mass calcd. for C₂₆H₃₄N₄O₂, 434.27; m/z found, 435.3 [M+H]⁺. ¹H NMR (CDCl₃): 8.37-8.34 (m, 1H), 8.34-8.32 (m, 1H), 7.55 (d, J=7.8, 1H), 7.24-7.17 (m, 3H), 6.92-6.90 (m, 1H), 3.71-3.68 (m, 2H), 3.49 (s, 2H), 3.44-3.39 (m, 2H), 2.93-2.90 (m, 1H), 2.82-2.79 (m, 1H), 2.75-2.71 (m, 2H), 2.45-2.30 (m, 4H), 1.92-1.87 (m, 1H), 1.87-1.76 (m, 1H), 1.74-1.69 (m, 1H), 1.52-1.47 (m, 4H), 1.42-1.36 (m, 2H), 0.49-0.32 (m, 4H).

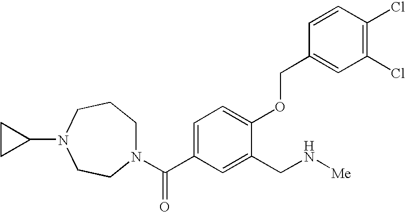

### Example 42

### (4-Cyclopropyl-[1,4]diazepan-1-yl)-[4-(3,4-dichloro-benzyloxy)-3-methylaminomethyl-phenyl]-methanone

Step A: [5-Bromo-2-(3,4-dichloro-benzyloxy)-benzyl]-methyl-carbamic acid tert-butyl ester. A mixture of (5-bromo-2-hydroxy-benzyl)-methyl-carbamic acid tert-butyl ester (1.0 g, 3.2 mmol), K₂CO₃ (655 mg, 4.74 mmol), and 4-bromomethyl-1,2-dichloro-benzene (1.14 g, 4.74 mmol) in CH₃CN (6.3 mL) was heated at 90° C. overnight. The mixture was then cooled to rt, diluted with EtOAc and water, and extracted with EtOAc (3×). The combined organic layers were washed with brine, dried (Na₂SO₄), and concentrated. Purification by FCC (EtOAc/hexanes) gave the desired product (1.49 g, 99%). ¹H NMR (CDCl₃, mixture of rotamers): 7.49 (d, J=1.9, 1H), 7.46 (d, J=7.8, 1H), 7.31 (dd, J=8.7, 2.0, 1H), 7.28-7.22 (m, 2H), 6.73 (d, J=8.6, 1H), 5.00 (s, 2H), 4.52-4.38 (m, 2H), 2.90-2.80 (m, 3H), 1.52-1.39 (m, 9H).

Step B: [5-(4-Cyclopropyl-[1,4]diazepane-1-carbonyl)-2-(3,4-dichloro-phenoxy)-benzyl]-methyl-carbamic acid tert-butyl ester. The title compound was prepared in an analogous fashion to Example 1, Step D. C₂₉H₃₇Cl₂N₃O₄, 561.2; m/z found, 562.3 [M+H]⁺. ¹H NMR (CDCl₃, mixture of rotamers): 7.46 (d, J=1.7, 1H), 7.40 (d, J=7.9, 1H), 7.29-7.24 (m, 1H), 7.21 (d, J=8.1, 1H), 7.11 (br s, 1H), 6.82 (d, J=8.3, 1H), 5.00 (s, 2H), 4.48-4.39 (m, 2H), 3.76-3.62 (m, 2H), 3.48-3.38 (m, 2H), 2.96-2.74 (m, 5H), 2.74-2.65 (m 2H), 1.93-1.71 (m, 2H), 1.67-1.57 (m, 1H), 1.46-1.32 (m, 9H), 0.44-0.30 (m, 4H).

Step C. The title compound was prepared in an analogous fashion to Example 1, Step E. MS (ESI): mass calcd. for C₂₄H₂₉Cl₂N₃O₂, 461.16; m/z found, 462.3 [M+H]⁺. ¹H NMR (CDCl₃): 7.51 (d, J=1.9, 1H), 7.46 (d, J=8.3, 1H), 7.36 (d, J=3.1, 1H), 7.28 (d, J=8.4, 1H), 7.24 (dd, J=8.3, 2.0, 1H), 6.85 (d, J=8.4, 1H), 5.07 (s, 2H), 3.81 (s, 2H), 3.76-3.67 (m, 2H), 3.54-3.42 (m, 2H), 2.96-2.94 (m, 1H), 2.86-2.81 (m, 1H), 2.81-2.72 (m, 2H), 2.44 (s, 3H), 1.98-1.71 (m, 3H), 1.66 (br s, 1H), 0.52-0.32 (m, 4H).

The compounds in Examples 43-54 were prepared using methods analogous to those described in the preceding examples.

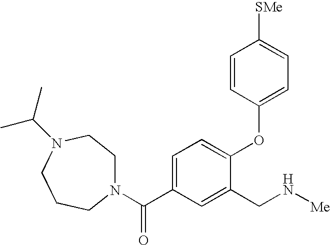

### Example 43

### (4-Isopropyl-[1,4]diazepan-1-yl)-[3-methylaminomethyl-4-(4-methylsulfanyl-phenoxy)-phenyl]-methanone

MS (ESI): m/z found, 428.4 [M+H]⁺.

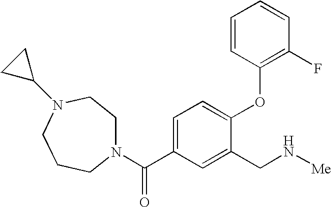

### Example 44

### (4-Cyclopropyl-[1,4]diazepan-1-yl)-[4-(2-fluoro-phenoxy)-3-methylaminomethyl-phenyl]-methanone

MS (ESI): m/z found, 398.8 [M+H]⁺.

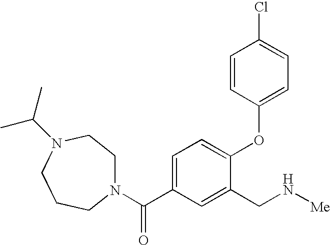

### Example 45

### [4-(4-Chloro-phenoxy)-3-methylaminomethyl-phenyl]-(4-isopropyl-[1,4]diazepan-1-yl)-methanone

MS (ESI): m/z found, 416.8 [M+H]⁺.

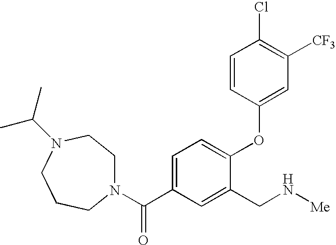

### Example 46

### [4-(4-Chloro-3-trifluoromethyl-phenoxy)-3-methylaminomethyl-phenyl]-(4-isopropyl-[1,4]diazepan-1-yl)-methanone

MS (ESI): m/z found, 484.8 [M+H]⁺.

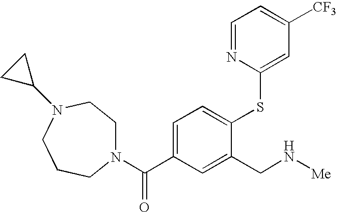

### Example 47

### (4-Cyclopropyl-[1,4]diazepan-1-yl)-[3-methylaminomethyl-4-(4-trifluoromethyl-pyridin-2-ylsulfanyl)-phenyl]-methanone

MS (ESI): m/z found, 465.8 [M+H]⁺.

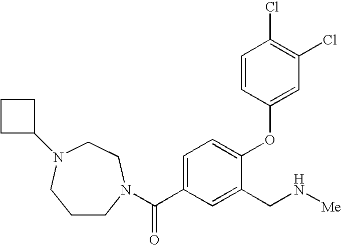

### Example 48

### (4-Cyclobutyl-[1,4]diazepan-1-yl)-[4-(3,4-dichloro-phenoxy)-3-methylaminomethyl-phenyl]-methanone

MS (ESI): m/z found, 462.2 [M+H]⁺.

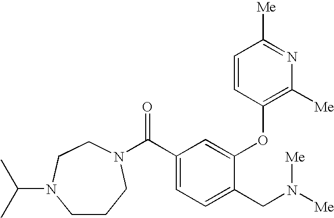

### Example 49

### [4-Dimethylaminomethyl-3-(2,6-dimethyl-pyridin-3-yloxy)-phenyl]-(4-isopropyl-[1,4]diazepan-1-yl)-methanone

MS (ESI): m/z found, 425.3 [M+H]⁺.

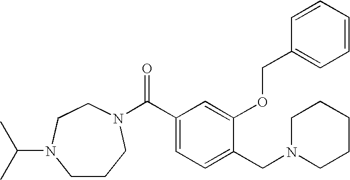

### Example 50

### (3-Benzyloxy-4-piperidin-1-ylmethyl-phenyl)-(4-isopropyl-[1,4]diazepan-1-yl)-methanone

MS (ESI): m/z found, 452.3 [M+H]⁺.

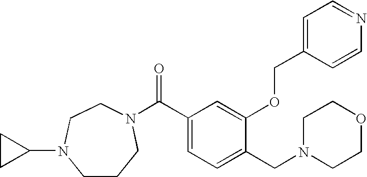

### Example 51

### (4-Cyclopropyl-[1,4]diazepan-1-yl)-[4-morpholin-4-ylmethyl-3-(pyridin-3-yloxy)-phenyl]-methanone

MS (ESI): m/z found, 437.3 [M+H]⁺.

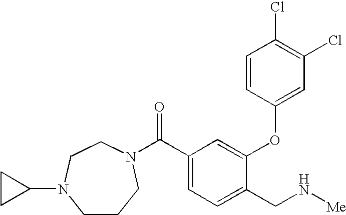

### Example 52

### (4-Cyclopropyl-[1,4]diazepan-1-yl)-[3-(3,4-dichloro-phenoxy)-4-methylaminomethyl-phenyl]-methanone

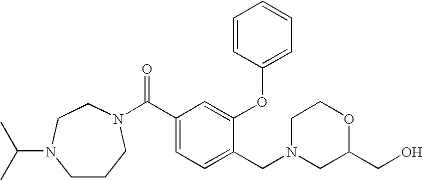

### Example 53

### [4-(2-Hydroxymethyl-morpholin-4-ylmethyl)-3-phenoxy-phenyl]-(4-isopropyl-[1,4]diazepan-1-yl)-methanone

MS (ESI): m/z found, 468.3 [M+H]⁺.

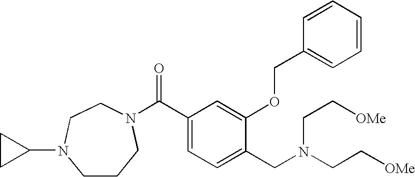

### Example 54

### (3-Benzyloxy-4-{[bis-(2-methoxy-ethyl)-amino]-methyl}-phenyl)-(4-cyclopropyl-[1,4]diazepan-1-yl)-methanone

MS (ESI): m/z found, 496.3 [M+H]⁺.

### Biological Methods:

Compounds were generally tested as the free base, hydrochloride salt, TFA salt, or citrate salt form.

### H₃ Receptor Binding

Binding of compounds to the cloned human and rat H₃ receptors, stably expressed in SK-N-MC cells, was performed as described by Barbier, A. J. et al. (Br. J. Pharmacol. 2004, 143(5), 649-661). Data for compounds tested in this assay are presented in Table 1.

### Rat Brain SERT

A rat brain without cerebellum (Zivic Laboratories, Inc.-Pittsburgh, Pa.) was homogenized in a 52.6 mM Tris pH 8/126.4 mM NaCl/5.26 mM KCl mixture and centrifuged at 1,000 rpm for 5 min. The supernatant was removed and re-centrifuged at 15,000 rpm for 30 min. Pellets were re-homogenized in a 52.6 mM Tris pH8/126.4 mM NaCl/5.26 mM KCl mixture. Membranes were incubated with 0.6 nM [³H]-Citalopram plus/minus test compounds for 60 min at 25° C. and harvested by rapid filtration over GF/C glass fiber filters (pretreated with 0.3% polyethylenimine) followed by four washes with ice-cold buffer. Nonspecific binding was defined in the presence of 100 μM fluoxetine. IC₅₀ values were determined by a single site curve-fitting program (GraphPad, San Diego, Calif.) and converted to Kᵢ values based on a [³H]-Citalopram K_{d} of 0.6 nM and a ligand concentration of 0.6 nM. Data for compounds tested in this assay are presented in Table 1. NT=not tested.

TABLE 1

| | Human H₃ | Rat SERT |
|---|---|---|
| Ex. | Kᵢ (nM) | Kᵢ (nM) |
| 1 | 1.8 | 9.1 |
| 2 | 2.0 | 11 |
| 3 | 1.2 | 4.4 |
| 4 | 0.5 | 22 |
| 5 | 1 | 4 |
| 6 | 1.0 | 39 |
| 7 | 0.7 | 3000 |
| 8 | 3 | 27 |
| 9 | 0.8 | 37 |
| 10 | 0.9 | 9.7 |
| 11 | 1.0 | 14 |
| 12 | 4.1 | 9.5 |
| 13 | 1.4 | 2.2 |
| 14 | 6 | 14 |
| 15 | 1.2 | 1.9 |
| 16 | 1 | 28 |
| 17 | 1.5 | 8.5 |
| 18 | 1.4 | 7.9 |
| 19 | 0.3 | NT |
| 20 | 1.1 | 24 |
| 21 | 0.9 | 34 |
| 22 | 0.7 | 13 |
| 23 | 1 | 20 |
| 24 | 0.8 | 6.7 |
| 25 | 0.6 | 61 |
| 26 | 1.0 | 4.5 |
| 27 | 5.5 | 11 |
| 28 | 2.1 | 299 |
| 29 | 1.0 | 238 |
| 30 | 2.0 | 900 |
| 31 | 2 | 28 |
| 32 | 0.6 | 31 |
| 33 | 0.9 | 28 |
| 34 | 2 | 62 |
| 35 | 2 | 15 |
| 36 | 2 | 59 |
| 37 | 2 | 5 |
| 38 | 2 | 5000 |
| 39 | 0.9 | 15 |
| 40 | 2 | 5000 |
| 41 | 1 | 10000 |
| 42 | 4 | 2 |
| 43 | 1.1 | 1.0 |
| 44 | 1.0 | 117 |
| 45 | 0.5 | 1.4 |
| 46 | 1 | 11 |
| 47 | 4 | 410 |
| 48 | 5.2 | 7.9 |
| 49 | 11 | 2000 |
| 50 | 9 | NT |
| 51 | 3 | NT |
| 52 | 6 | 17 |
| 53 | 3 | NT |
| 54 | 2 | NT |

### Human SERT

Homogenized HEK293 (Human Embryonic Kidney) membranes expressing the human SERT were incubated with ³H-citalopram (SERT) at rt for 1 h in 50 mM Tris, 120 mM NaCl, 5 mM KCl (pH 7.4). Nonspecific binding was determined in the presence of 10 μM fluoxetine for the SERT. The membranes were washed and the radioactivity was counted as above. Calculations for Kᵢ at the SERT were based on a K_{d} value for ³H-citalopram and a ligand concentration of 3.1 nM. Data for compounds tested in this assay are presented in Table 2.

TABLE 2

| | Human SERT |
|---|---|
| Ex. | Kᵢ (nM) |
| 1 | 9.0 |
| 2 | 17 |
| 3 | 19 |
| 4 | 120 |
| 5 | 8 |
| 6 | 236 |
| 7 | 1130 |
| 8 | 79 |
| 9 | 229 |
| 10 | 47 |
| 11 | 116 |
| 12 | 12 |
| 13 | 2.9 |
| 15 | 14 |
| 17 | 36 |
| 18 | 41 |
| 20 | 94 |
| 22 | 122 |
| 24 | 68 |
| 26 | 13 |
| 27 | 95 |
| 28 | 702 |
| 29 | 797 |
| 30 | 1400 |
| 43 | 5.0 |
| 44 | 562 |
| 45 | 6.4 |
| 48 | 9.8 |

### Cyclic AMP Accumulation

Sublines of SK-N-MC cells were created that expressed a reporter construct and the human H₃ receptor. The reporter gene (β-galactosidase) is under the control of multiple cyclic AMP responsive elements. In 96-well plates, histamine was added directly to the cell media followed 5 min later by an addition of forskolin (5 μM final concentration). When appropriate, antagonists were added 10 min prior to agonist addition. After a 6-h incubation at 37° C., the media was aspirated and the cells washed with 200 μL of phosphate-buffered saline followed by a second aspiration. Cells were lysed with 25 μL 0.1× assay buffer (10 mM Na-phosphate, pH 8, 0.2 mM MgSO₄, 0.01 mM MnCl₂) and incubated at rt for 10 min. Cells were then incubated for 10 min with 100 μL of 1× assay buffer containing 0.5% Triton and 40 mM β-mercaptoethanol. Color was developed using 25 μL of 1 mg/mL substrate solution (chlorophenolred β-D galactopyranoside; Roche Molecular Biochemicals, Indianapolis, Ind.). Color was quantitated on a microplate reader at absorbance 570 nM. The pA₂ values were calculated by Schild regression analysis of the pEC₅₀ values and are presented for compounds tested in Table 3.

TABLE 3

| Ex. | pA₂ |
|---|---|
| 1 | 9.76 |
| 2 | 9.68 |
| 3 | 9.27 |
| 4 | 10.3 |
| 5 | 10.2 |
| 6 | 10.57 |
| 7 | 10.5 |
| 9 | 10.7 |
| 10 | 10.4 |
| 11 | 9.98 |
| 12 | 9.06 |
| 15 | 8.87 |
| 20 | 10.3 |
| 22 | 10.44 |
| 26 | 10.37 |
| 27 | 9.23 |
| 28 | 9.60 |
| 29 | 10.37 |
| 30 | 9.41 |
| 40 | 9.3 |
| 41 | 10.2 |

What is claimed is:

1. A compound of Formula (I): 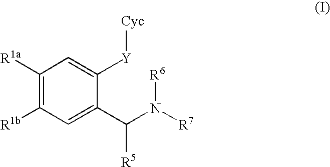
wherein
one of R^{1a} and R^{1b} is 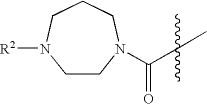
and the other is —H;
Y is —O—, —OCH₂—, —S—, —SO—, or —SO₂—;
R² is —H; a —C₁₋₆alkyl group unsubstituted or substituted with —OH, —OC₁₋₄alkyl, —NH₂, —NH(C₁₋₄alkyl), —N(C₁₋₄alkyl)₂, or —F; —CO₂C₁₋₄alkyl; or a monocyclic cycloalkyl group unsubstituted or substituted with —C₁₋₄alkyl, —OH, halo, or —CF₃;
R⁵ is —H or —C₁₋₆alkyl;
R⁶ is —H; or —C₁₋₆alkyl, —C₃₋₆alkenyl, —C₃₋₆alkynyl, monocyclic cycloalkyl, or —C₁₋₆alkyl-(monocyclic cycloalkyl), each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
R⁷ is —H; or —C₁₋₆alkyl, —C₃₋₆alkenyl, —C₃₋₆alkynyl, monocyclic cycloalkyl, —C₁₋₆alkyl-(monocyclic cycloalkyl), or —CO₂C₁₋₆alkyl, each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
or R⁶ and R⁷ taken together with their nitrogen of attachment form a saturated monocyclic heterocycloalkyl group unsubstituted or substituted with —C₁₋₄alkyl, —OH, —C₁₋₄alkyl-OH, —OC₁₋₄alkyl, or halo; and
Cyc is a phenyl or monocyclic carbon-linked heteroaryl group, unsubstituted or substituted with one, two, or three R^{k} moieties;
where each R^{k} moiety is independently selected from the group consisting of: —C₁₋₆alkyl, —CHF₂, —CF₃, —C₂₋₆alkenyl, —C₂₋₆alkynyl, —OH, —OC₁₋₆alkyl, —OCHF₂, —OCF₃, —OC₃₋₆alkenyl, —OC₃₋₆alkynyl, —CN, —NO₂, —N(R^{l})R^{m}, —N(R^{l})C(O)R^{m}, —N(R^{l})SO₂C₁₋₆alkyl, —C(O)C₁₋₆alkyl, —S(O)₀₋₂—C₁₋₆alkyl, —C(O)N(R^{l})R^{m}, —SO₂N(R^{l})R^{m}, —SCF₃, halo, —CO₂H, and —CO₂C₁₋₆alkyl; or two R^{k} moieties on adjacent carbon atoms of attachment together are —OC₁₋₄alkyleneO— to form a cyclic ring which is unsubstituted or substituted with one or two fluoro substituents;
where R^{l} and R^{m} are each independently —H or —C₁₋₆alkyl;
or a pharmaceutically acceptable salt, a pharmaceutically acceptable prodrug, or a pharmaceutically active metabolite thereof.

2. A compound as defined in claim 1, wherein R^{1a} is

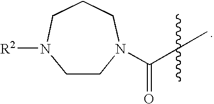

3. A compound as defined in claim 1, wherein Y is —O—.

4. A compound as defined in claim 1, wherein Y is —S—.

5. A compound as defined in claim 1, wherein R² is —H; or methyl, ethyl, propyl, isopropyl, sec-butyl, 2-methylpropyl, cyclopropyl, cyclobutyl, or cyclopentyl, each unsubstituted or substituted as previously described.

6. A compound as defined in claim 1, wherein R² is —H, methyl, ethyl, propyl, isopropyl, sec-butyl, 2-hydroxyethyl, 2-methoxyethyl, 2-dimethylaminoethyl, 2-hydroxy-2-methylpropyl, 3-dimethylaminopropyl, cyclopropyl, cyclobutyl, or cyclopentyl.

7. A compound as defined in claim 1, wherein R² is —H, methyl, or cyclopropyl.

8. A compound as defined in claim 1, wherein R⁵ is —H or methyl.

9. A compound as defined in claim 1, wherein R⁵ is —H.

10. A compound as defined in claim 1, wherein R⁶ is —H, methyl, ethyl, isopropyl, sec-butyl, cyclopropyl, cyclobutyl, or cyclopentyl, each unsubstituted or substituted as previously described.

11. A compound as defined in claim 1, wherein R⁶ is —H, methyl, or methoxyethyl.

12. A compound as defined in claim 1, wherein R⁷ is —H, methyl, ethyl, propyl, isopropyl, sec-butyl, cyclopropyl, cyclobutyl, cyclopentyl, cyclohexyl, cyclopropylmethyl, cyclobutylmethyl, cyclopentylmethyl, or tert-butoxycarbonyl, each unsubstituted or substituted as previously described.

13. A compound as defined in claim 1, wherein R⁷ is methyl, ethyl, methoxyethyl, isopropyl, sec-butyl, cyclopropyl, cyclobutyl, or cyclopentyl.

14. A compound as defined in claim 1, wherein R⁷ is methyl or cyclopropyl.

15. A compound as defined in claim 1, wherein R⁶ and R⁷ taken together with their nitrogen of attachment form azetidinyl, pyrrolidinyl, piperidinyl, piperazinyl, morpholinyl, thiomorpholinyl, 1,1-dioxo-1λ⁶-thiomorpholin-4-yl, homopiperidinyl, diazepanyl, or homomorpholinyl, each unsubstituted or substituted as previously described.

16. A compound as defined in claim 1, wherein R⁶ and R⁷ taken together with their nitrogen of attachment form piperidinyl, pyrrolidinyl, morpholinyl, 2-hydroxymethyl-morpholin-4-yl, or homomorpholinyl.

17. A compound as defined in claim 1, wherein Cyc is a phenyl or pyridyl group unsubstituted or substituted with one, two, or three R^{k} moieties.

18. A compound as defined in claim 1, wherein Cyc is a thiophenyl, oxazolyl, thiazolyl, pyrazolyl, pyridinyl, or pyrazinyl group unsubstituted or substituted with one, two, or three R^{k} moieties.

19. A compound as defined in claim 1, wherein Cyc is phenyl, 2-hydroxyphenyl, 3-hydroxyphenyl, 4-hydroxyphenyl, 4-hydroxy-2-methylphenyl, 4-hydroxy-3-fluorophenyl, 3,4-dihydroxyphenyl, 2-methoxyphenyl, 3-methoxyphenyl, 4-methoxyphenyl, 4-ethoxyphenyl, 2,4-dimethoxyphenyl, 2,5-dimethoxyphenyl, 3,4-dimethoxyphenyl, 3,5-dimethoxyphenyl, 3,4,5-trimethoxyphenyl, 2-methylphenyl, 3-methylphenyl, 4-methylphenyl, 4-ethylphenyl, 3-ethynylphenyl, 4-ethynylphenyl, 2-chlorophenyl, 3-chlorophenyl, 4-chlorophenyl, 2-fluorophenyl, 3-fluorophenyl, 4-fluorophenyl, 2-bromophenyl, 3-bromophenyl, 4-bromophenyl, 3-iodophenyl, 4-iodophenyl, 2,3-difluorophenyl, 2,4-difluorophenyl, 3,4-difluorophenyl, 2,3-dichlorophenyl, 2,4-dichlorophenyl, 2,5-dichlorophenyl, 3,4-dichlorophenyl, 3,5-dichlorophenyl, 2-fluoro-3-chlorophenyl, 2-fluoro-4-chlorophenyl, 2-chloro-4-fluorophenyl, 3-fluoro-4-chlorophenyl, 3-chloro-4-fluorophenyl, 4-fluoro-3-methylphenyl, 3-chloro-4-methoxyphenyl, 2-fluoro-4-methoxyphenyl, 3-fluoro-4-methoxyphenyl, 3-chloro-4-difluoromethoxyphenyl, 4-chloro-3-trifluoromethylphenyl, 2-trifluoromethylphenyl, 3-trifluoromethylphenyl, 4-trifluoromethylphenyl, 3-trifluoromethoxyphenyl, 4-trifluoromethoxyphenyl, 4-difluoromethoxyphenyl, 2-cyanophenyl, 3-cyanophenyl, 4-cyanophenyl, 3-acetylphenyl, 4-acetylphenyl, 3-nitrophenyl, 4-nitrophenyl, 4-aminophenyl, 4-dimethylaminophenyl, 4-carbamoylphenyl, 4-methanesulfanylphenyl, 4-methanesulfinylphenyl, 4-methanesulfonylphenyl, 4-trifluoromethanesulfanylphenyl, 3-methyl-4-methylsulfanylphenyl, benzo[1,3]dioxol-4-yl, benzo[1,3]dioxol-5-yl, thiophen-2-yl, thiophen-3-yl, oxazol-5-yl, thiazol-5-yl, thiazol-2-yl, 2H-pyrazol-3-yl, 2-pyridinyl, 3-pyridinyl, 4-pyridinyl, 4-trifluoromethyl-pyridin-2-yl, 2,6-dimethyl-pyridin-3-yl, 6-methyl-pyridin-3-yl, 2-chloro-5-pyridinyl, 2-dimethylamino-5-pyridinyl, 6-methoxy-pyridin-3-yl, 6-methylsulfanyl-pyridin-3-yl, 2-hydroxy-5-pyridinyl, 6-bromo-pyridin-3-yl, or pyrazin-2-yl.

20. A compound as defined in claim 1, wherein Cyc is phenyl, 3-methoxyphenyl, 2-trifluoromethoxyphenyl, 2-fluorophenyl, 3-fluorophenyl, 4-fluorophenyl, 3-chlorophenyl, 4-chlorophenyl, 2,3-difluorophenyl, 2,3-dichlorophenyl, 3,4-dichlorophenyl, 2-chloro-4-fluorophenyl, 3-chloro-2-fluorophenyl, 4-chloro-2-fluorophenyl, 2-trifluoromethylphenyl, 4-trifluoromethylphenyl, 4-chloro-3-trifluoromethylphenyl, 4-methanesulfanylphenyl, 3-methyl-4-methanesulfanylphenyl, 4-trifluoromethanesulfanylphenyl, 4-trifluoromethyl-pyridin-2-yl, 2,6-dimethyl-pyridin-3-yl, 2-cyanophenyl, 3-cyanophenyl, 4-cyanophenyl, 2-pyridinyl, 3-pyridinyl, or 6-methyl-3-pyridinyl.

21. A compound as defined in claim 1, wherein each R^{k} moiety is selected from the group consisting of: methyl, fluoro, chloro, trifluoromethyl, methanesulfanyl, trifluoromethanesulfanyl, cyano, methoxy, and trifluoromethoxy.

22. A compound as defined in claim 1, wherein R^{l} and R^{m} are each independently —H or methyl.

23. A compound selected from the group consisting of:
(4-Cyclopropyl-[1,4]diazepan-1-yl)-[4-(3,4-dichloro-phenoxy)-3-methylaminomethyl-phenyl]-methanone;
[4-(4-Chloro-phenoxy)-3-methylaminomethyl-phenyl]-(5-isopropyl-2,5-diaza-bicyclo[2.2.1]hept-2-yl)-methanone;
(4-Cyclopropyl-[1,4]diazepan-1-yl)-[3-methylaminomethyl-4-(4-trifluoromethyl-phenoxy)-phenyl]-methanone;
(4-Isopropyl-[1,4]diazepan-1-yl)-[3-methylaminomethyl-4-(4-trifluoromethyl-phenoxy)-phenyl]-methanone;
(4-Cyclopropyl-[1,4]diazepan-1-yl)-[3-methylaminomethyl-4-(pyridin-3-yloxy)-phenyl]-methanone;
[4-(4-Chloro-phenoxy)-3-methylaminomethyl-phenyl]-(4-cyclopropyl-[1,4]diazepan-1-yl)-methanone;
(4-Cyclopropyl-[1,4]diazepan-1-yl)-[4-(3-fluoro-phenoxy)-3-methylaminomethyl-phenyl]-methanone;
[3-Cyclopropylaminomethyl-4-(pyridin-3-yloxy)-phenyl]-(4-cyclopropyl-[1,4]diazepan-1-yl)-methanone;
[4-(4-Chloro-phenoxy)-3-cyclopropylaminomethyl-phenyl]-(4-cyclopropyl-[1,4]diazepan-1-yl)-methanone;
(4-Cyclopropyl-[1,4]diazepan-1-yl)-(3-methylaminomethyl-4-phenoxy-phenyl)-methanone;
[4-(3-Chloro-phenoxy)-3-methylaminomethyl-phenyl]-(4-cyclopropyl-[1,4]diazepan-1-yl)-methanone;
[4-(3-Chloro-phenoxy)-3-methylaminomethyl-phenyl]-(4-isopropyl-[1,4]diazepan-1-yl)-methanone;
(4-Cyclopropyl-[1,4]diazepan-1-yl)-[3-methylaminomethyl-4-(3-methyl-4-methylsulfanyl-phenoxy)-phenyl]-methanone;
(4-Cyclopropyl-[1,4]diazepan-1-yl)-[3-methylaminomethyl-4-(4-methylsulfanyl-phenoxy)-phenyl]-methanone;
[3-Cyclopropylaminomethyl-4-(3,4-dichloro-phenoxy)-phenyl]-(4-cyclopropyl-[1,4]diazepan-1-yl)-methanone;
[4-(3,4-Dichloro-phenoxy)-3-methylaminomethyl-phenyl]-(4-isopropyl-[1,4]diazepan-1-yl)-methanone;
[4-(3-Chloro-2-fluoro-phenoxy)-3-methylaminomethyl-phenyl]-(4-cyclopropyl-[1,4]diazepan-1-yl)-methanone;
[4-(3-Chloro-4-fluoro-phenoxy)-3-methylaminomethyl-phenyl]-(4-cyclopropyl-[1,4]diazepan-1-yl)-methanone;
[4-(4-Chloro-2-fluoro-phenoxy)-3-methylaminomethyl-phenyl]-(4-cyclopropyl-[1,4]diazepan-1-yl)-methanone;
(4-Cyclopropyl-[1,4]diazepan-1-yl)-[3-methylaminomethyl-4-(6-methyl-pyridin-3-yloxy)-phenyl]-methanone;
4-[4-(4-Cyclopropyl-[1,4]diazepane-1-carbonyl)-2-methylaminomethyl-phenoxy]-benzonitrile;
[4-(4-Chloro-phenoxy)-3-cyclopropylaminomethyl-phenyl]-(4-isopropyl-[1,4]diazepan-1-yl)-methanone;
3-[4-(4-Cyclopropyl-[1,4]diazepane-1-carbonyl)-2-methylaminomethyl-phenoxy]-benzonitrile;
[3-Cyclopropylaminomethyl-4-(3,4-dichloro-phenoxy)-phenyl]-(4-isopropyl-[1,4]diazepan-1-yl)-methanone;
(4-Cyclopropyl-[1,4]diazepan-1-yl)-[4-(4-fluoro-phenoxy)-3-methylaminomethyl-phenyl]-methanone;
[4-(2-Chloro-4-fluoro-phenoxy)-3-methylaminomethyl-phenyl]-(4-isopropyl-[1,4]diazepan-1-yl)-methanone;
(4-Cyclopropyl-[1,4]diazepan-1-yl)-[4-(3-methoxy-phenoxy)-3-methylaminomethyl-phenyl]-methanone;
(4-Cyclopropyl-[1,4]diazepan-1-yl)-[3-methylaminomethyl-4-(4-trifluoromethylsulfanyl-phenoxy)-phenyl]-methanone;
(4-Cyclopropyl-[1,4]diazepan-1-yl)-[3-methylaminomethyl-4-(2-trifluoromethoxy-phenoxy)-phenyl]-methanone;
2-[4-(4-Cyclopropyl-[1,4]diazepane-1-carbonyl)-2-methylaminomethyl-phenoxy]-benzonitrile;
(4-Cyclopropyl-[1,4]diazepan-1-yl)-[3-methylaminomethyl-4-(2-trifluoromethyl-phenoxy)-phenyl]-methanone;
[4-(4-Chloro-3-trifluoromethyl-phenoxy)-3-methylaminomethyl-phenyl]-(4-cyclopropyl-[1,4]diazepan-1-yl)-methanone;
(4-Cyclopropyl-[1,4]diazepan-1-yl)-[4-(2,3-difluoro-phenoxy)-3-methylaminomethyl-phenyl]-methanone;
[4-(2-Chloro-4-fluoro-phenoxy)-3-methylaminomethyl-phenyl]-(4-cyclopropyl-[1,4]diazepan-1-yl)-methanone;
{4-(4-Chloro-phenoxy)-3-[(cyclopropyl-methyl-amino)-methyl]-phenyl}-(4-cyclopropyl-[1,4]diazepan-1-yl)-methanone;
(4-Cyclopropyl-[1,4]diazepan-1-yl)-[4-(2,3-dichloro-phenoxy)-3-methylaminomethyl-phenyl]-methanone;
(4-Cyclopropyl-[1,4]diazepan-1-yl)-[3-dimethylaminomethyl-4-(6-methyl-pyridin-3-yloxy)-phenyl]-methanone;
[4-(4-Chloro-phenylsulfanyl)-3-methylaminomethyl-phenyl]-(5-isopropyl-2,5-diaza-bicyclo[2.2.1]hept-2-yl)-methanone;
[4-(4-Chloro-phenylsulfanyl)-3-methylaminomethyl-phenyl]-(4-cyclopropyl-[1,4]diazepan-1-yl)-methanone;
(4-Cyclopropyl-[1,4]diazepan-1-yl)-[3-methylaminomethyl-4-(pyridin-2-ylsulfanyl)-phenyl]-methanone;
(4-Cyclopropyl-[1,4]diazepan-1-yl)-[3-methylaminomethyl-4-(pyridin-2-yloxy)-phenyl]-methanone;
[4-Cyclopropylaminomethyl-3-(pyridin-3-yloxy)-phenyl]-(4-cyclopropyl-[1,4]diazepan-1-yl)-methanone;
(4-Cyclopropyl-[1,4]diazepan-1-yl)-[4-piperidin-1-ylmethyl-3-(pyridin-3-yloxy)-phenyl]-methanone;
(4-Cyclopropyl-[1,4]diazepan-1-yl)-[4-(3,4-dichloro-benzyloxy)-3-methylaminomethyl-phenyl]-methanone;
(4-Isopropyl-[1,4]diazepan-1-yl)-[3-methylaminomethyl-4-(4-methylsulfanyl-phenoxy)-phenyl]-methanone;
(4-Cyclopropyl-[1,4]diazepan-1-yl)-[4-(2-fluoro-phenoxy)-3-methylaminomethyl-phenyl]-methanone;
[4-(4-Chloro-phenoxy)-3-methylaminomethyl-phenyl]-(4-isopropyl-[1,4]diazepan-1-yl)-methanone;
[4-(4-Chloro-3-trifluoromethyl-phenoxy)-3-methylaminomethyl-phenyl]-(4-isopropyl-[1,4]diazepan-1-yl)-methanone;
(4-Cyclopropyl-[1,4]diazepan-1-yl)-[3-methylaminomethyl-4-(4-trifluoromethyl-pyridin-2-ylsulfanyl)-phenyl]-methanone;
(4-Cyclobutyl-[1,4]diazepan-1-yl)-[4-(3,4-dichloro-phenoxy)-3-methylaminomethyl-phenyl]-methanone;
[4-Dimethylaminomethyl-3-(2,6-dimethyl-pyridin-3-yloxy)-phenyl]-(4-isopropyl-[1,4]diazepan-1-yl)-methanone;
(3-Benzyloxy-4-piperidin-1-ylmethyl-phenyl)-(4-isopropyl-[1,4]diazepan-1-yl)-methanone;
(4-Cyclopropyl-[1,4]diazepan-1-yl)-[4-morpholin-4-ylmethyl-3-(pyridin-3-yloxy)-phenyl]-methanone;
(4-Cyclopropyl-[1,4]diazepan-1-yl)-[3-(3,4-dichloro-phenoxy)-4-methylaminomethyl-phenyl]-methanone;
[4-(2-Hydroxymethyl-morpholin-4-ylmethyl)-3-phenoxy-phenyl]-(4-isopropyl-[1,4]diazepan-1-yl)-methanone; and
(3-Benzyloxy-4-{[bis-(2-methoxy-ethyl)-amino]-methyl}-phenyl)-(4-cyclopropyl-[1,4]diazepan-1-yl)-methanone;
and pharmaceutically acceptable salts thereof.

24. A compound or pharmaceutically acceptable salt according to claim 1.

25. A compound of Formula (II): 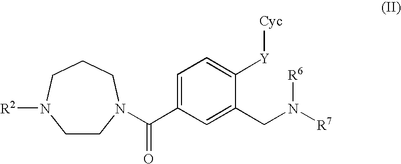
wherein
Y is —O— or —S—;
R² is —H; a —C₁₋₆alkyl group unsubstituted or substituted with —OH, —OC₁₋₄alkyl, —NH₂, —NH(C₁₋₄alkyl), —N(C₁₋₄alkyl)₂, or —F; —CO₂C₁₋₄alkyl; or a monocyclic cycloalkyl group unsubstituted or substituted with —C₁₋₄alkyl, —OH, halo, or —CF₃;
R⁶ is —H; or —C₁₋₆alkyl, —C₃₋₆alkenyl, —C₃₋₆alkynyl, monocyclic cycloalkyl, or —C₁₋₆alkyl-(monocyclic cycloalkyl), each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
R⁷ is —H; or —C₁₋₆alkyl, —C₃₋₆alkenyl, —C₃₋₆alkynyl, monocyclic cycloalkyl, —C₁₋₆alkyl-(monocyclic cycloalkyl), or —CO₂C₁₋₆alkyl, each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
or R⁶ and R⁷ taken together with their nitrogen of attachment form a saturated monocyclic heterocycloalkyl group unsubstituted or substituted with —C₁₋₄alkyl, —OH, —C₁₋₄alkyl-OH, —OC₁₋₄alkyl, or halo; and
Cyc is a phenyl or monocyclic carbon-linked heteroaryl group, unsubstituted or substituted with one, two, or three R^{k} moieties;
where each R^{k} moiety is independently selected from the group consisting of: —C₁₋₆alkyl, —CHF₂, —CF₃, —C₂₋₆alkenyl, —C₂₋₆alkynyl, —OH, —OC₁₋₆alkyl, —OCHF₂, —OCF₃, —OC₃₋₆alkenyl, —OC₃₋₆alkynyl, —CN, —NO₂, —N(R^{l})R^{m}, —N(R^{l})C(O)R^{m}, —N(R^{l})SO₂C₁₋₆alkyl, —C(O)C₁₋₆alkyl, —S(O)₀₋₂—C₁₋₆alkyl, —C(O)N(R^{l})R^{m}, —SO₂N(R^{l})R^{m}, —SCF₃, halo, —CO₂H, and —CO₂C₁₋₆alkyl; or two R^{k} moieties on adjacent carbon atoms of attachment together are —OC₁₋₄alkyleneO— to form a cyclic ring which is unsubstituted or substituted with one or two fluoro substituents;
where R^{l} and R^{m} are each independently —H or —C₁₋₆alkyl;
or a pharmaceutically acceptable salt, a pharmaceutically acceptable prodrug, or a pharmaceutically active metabolite thereof.

26. A compound as defined in claim 25, wherein Cyc is a phenyl or pyridyl group unsubstituted or substituted with one, two, or three R^{k} moieties.

27. A pharmaceutical composition for treating a disease, disorder, or medical condition mediated by histamine H₃ receptor and/or serotonin transporter activity, comprising:
(a) an effective amount of a compound of Formula (I): 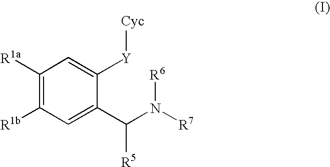
wherein
one of R^{1a} and R^{1b} is 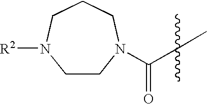
and the other is —H;
Y is —O—, —OCH₂—, —S—, —SO—, or —SO₂—;
R² is —H; a —C₁₋₆alkyl group unsubstituted or substituted with —OH, —OC₁₋₄alkyl, —NH₂, —NH(C₁₋₄alkyl), —N(C₁₋₄alkyl)₂, or —F; —CO₂C₁₋₄alkyl; or a monocyclic cycloalkyl group unsubstituted or substituted with —C₁₋₄alkyl, —OH, halo, or —CF₃;
R⁵ is —H or —C₁₋₆alkyl;
R⁶ is —H; or —C₁₋₆alkyl, —C₃₋₆alkenyl, —C₃₋₆alkynyl, monocyclic cycloalkyl, or —C₁₋₆alkyl-(monocyclic cycloalkyl), each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
R⁷ is —H; or —C₁₋₆alkyl, —C₃₋₆alkenyl, —C₃₋₆alkynyl, monocyclic cycloalkyl, —C₁₋₆alkyl-(monocyclic cycloalkyl), or —CO₂C₁₋₆alkyl, each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
or R⁶ and R⁷ taken together with their nitrogen of attachment form a saturated monocyclic heterocycloalkyl group unsubstituted or substituted with —C₁₋₄alkyl, —OH, —C₁₋₄alkyl-OH, —OC₁₋₄alkyl, or halo; and
Cyc is a phenyl or monocyclic carbon-linked heteroaryl group, unsubstituted or substituted with one, two, or three R^{k} moieties;
where each R^{k} moiety is independently selected from the group consisting of: —C₁₋₆alkyl, —CHF₂, —CF₃, —C₂₋₆alkenyl, —C₂₋₆alkynyl, —OH, —OC₁₋₆alkyl, —OCHF₂, —OCF₃, —OC₃₋₆alkenyl, —OC₃₋₆alkynyl, —CN, —NO₂, —N(R^{l})R^{m}, —N(R^{l})C(O)R^{m}, —N(R^{l})SO₂C₁₋₆alkyl, —C(O)C₁₋₆alkyl, —S(O)₀₋₂—C₁₋₆alkyl, —C(O)N(R^{l})R^{m}, —SO₂N(R^{l})R^{m}, —SCF₃, halo, —CO₂H, and —CO₂C₁₋₆alkyl; or two R^{k} moieties on adjacent carbon atoms of attachment together are —OC₁₋₄alkyleneO— to form a cyclic ring which is unsubstituted or substituted with one or two fluoro substituents;
where R^{l} and R^{m} are each independently —H or —C₁₋₆alkyl;
or a pharmaceutically acceptable salt, pharmaceutically acceptable prodrug, or pharmaceutically active metabolite thereof; and
(b) a pharmaceutically acceptable excipient.

28. A pharmaceutical composition according to claim 27, further comprising: an active ingredient selected from the group consisting of H₁ receptor antagonists, H₂ receptor antagonists, H₃ receptor antagonists, serotonin-norepinephrine reuptake inhibitors, selective serotonin reuptake inhibitors, noradrenergic reuptake inhibitors, non-selective serotonin re-uptake inhibitors, acetylcholinesterase inhibitors, and modafinil.

29. A method of treating a subject suffering from or diagnosed with a disease, disorder, or medical condition mediated by histamine H₃ receptor and/or serotonin transporter activity, comprising administering to the subject in need of such treatment an effective amount of a compound of Formula (I): 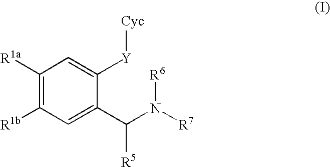
wherein
one of R^{1a} and R^{1b} is 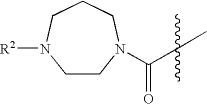
and the other is —H;
Y is —O—, —OCH₂—, —S—, —SO—, or —SO₂—;
R² is —H; a —C₁₋₆alkyl group unsubstituted or substituted with —OH, —OC₁₋₄alkyl, —NH₂, —NH(C₁₋₄alkyl), —N(C₁₋₄alkyl)₂, or —F; —CO₂C₁₋₄alkyl; or a monocyclic cycloalkyl group unsubstituted or substituted with —C₁₋₄alkyl, —OH, halo, or —CF₃;
R⁵ is —H or —C₁₋₆alkyl;
R⁶ is —H; or —C₁₋₆alkyl, —C₃₋₆alkenyl, —C₃₋₆alkynyl, monocyclic cycloalkyl, or —C₁₋₆alkyl-(monocyclic cycloalkyl), each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
R⁷ is —H; or —C₁₋₆alkyl, —C₃₋₆alkenyl, —C₃₋₆alkynyl, monocyclic cycloalkyl, —C₁₋₆alkyl-(monocyclic cycloalkyl), or —CO₂C₁₋₆alkyl, each unsubstituted or substituted with —C₁₋₄alkyl, —OH, —OC₁₋₄alkyl, halo, —NH₂, —NH(C₁₋₄alkyl), —N(C₁₋₄alkyl)₂, —CN, —CO₂H, or —CO₂C₁₋₄alkyl;
or R⁶ and R⁷ taken together with their nitrogen of attachment form a saturated monocyclic heterocycloalkyl group unsubstituted or substituted with —C₁₋₄alkyl, —OH, —C₁₋₄alkyl-OH, —OC₁₋₄alkyl, or halo; and
Cyc is a phenyl or monocyclic carbon-linked heteroaryl group, unsubstituted or substituted with one, two, or three R^{k} moieties;
where each R^{k} moiety is independently selected from the group consisting of: —C₁₋₆alkyl, —CHF₂, —CF₃, —C₂₋₆alkenyl, —C₂₋₆alkynyl, —OH, —OC₁₋₆alkyl, —OCHF₂, —OCF₃, —OC₃₋₆alkenyl, —OC₃₋₆alkynyl, —CN, —NO₂, —N(R^{l})R^{m}, —N(R^{l})C(O)R^{m}, —N(R^{l})SO₂C₁₋₆alkyl, —C(O)C₁₋₆alkyl, —S(O)₀₋₂—C₁₋₆alkyl, —C(O)N(R^{l})R^{m}, —SO₂N(R^{l})R^{m}, —SCF₃, halo, —CO₂H, and —CO₂C₁₋₆alkyl; or two R^{k} moieties on adjacent carbon atoms of attachment together are —OC₁₋₄alkyleneO— to form a cyclic ring which is unsubstituted or substituted with one or two fluoro substituents;
where R^{l} and R^{m} are each independently —H or —C₁₋₆alkyl;
or a pharmaceutically acceptable salt, pharmaceutically acceptable prodrug, or pharmaceutically active metabolite thereof.

30. The method according to claim 29, wherein the disease, disorder, or medical condition is selected from the group consisting of: cognitive disorders, sleep disorders, psychiatric disorders, and other disorders.

31. The method according to claim 29, wherein the disease, disorder, or medical condition is selected from the group consisting of: dementia, Alzheimer's disease, cognitive dysfunction, mild cognitive impairment, pre-dementia, attention deficit hyperactivity disorders, attention-deficit disorders, and learning and memory disorders.

32. The method according to claim 29, wherein the disease, disorder, or medical condition is selected from the group consisting of: learning impairment, memory impairment, and memory loss.

33. The method according to claim 29, wherein the disease, disorder, or medical condition is selected from the group consisting of: insomnia, disturbed sleep, narcolepsy with or without associated cataplexy, cataplexy, disorders of sleep/wake homeostasis, idiopathic somnolence, excessive daytime sleepiness, circadian rhythm disorders, fatigue, lethargy, and jet lag.

34. The method according to claim 29, wherein the disease, disorder, or medical condition is selected from the group consisting of: sleep apnea, perimenopausal hormonal shifts, Parkinson's disease, multiple sclerosis, depression, chemotherapy, and shift work schedules.

35. The method according to claim 29, wherein the disease, disorder, or medical condition is selected from the group consisting of: schizophrenia, bipolar disorders, manic disorders, depression, obsessive-compulsive disorder, and post-traumatic stress disorder.

36. The method according to claim 29, wherein the disease, disorder, or medical condition is selected from the group consisting of: motion sickness, vertigo, epilepsy, migraine, neurogenic inflammation, eating disorders, obesity, and substance abuse disorders.

37. The method according to claim 29, wherein the disease, disorder, or medical condition is selected from the group consisting of: depression, disturbed sleep, fatigue, lethargy, cognitive impairment, memory impairment, memory loss, learning impairment, attention-deficit disorders, and eating disorders.

38. A pharmaceutical composition according to claim 27, further comprising topiramate.

39. The method according to claim 29, wherein the disease, disorder, or medical condition is selected from the group consisting of: age-related cognitive decline, REM-behavioral disorder, benign postural vertigo, tinitus, movement disorders, restless leg syndrome, eye-related disorders, macular degeneration, and retinitis pigmentosis.
